(19) United States
(12) Patent Application Publication
(10) Pub. No.: US 2026/0341407 A1
(43) Pub. Date: Oct. 29, 2026

(54) TUMOR-SPECIFIC METHYLATION-BASED MULTI-OMIC METHOD FOR DETECTING GENE DELETIONS AND DRIVER MUTATIONS FROM PLASMA AND TISSUE DNA

(71) Applicant: GUARDANT HEALTH, INC., Palo Alto, CA (US)

(72) Inventors: Sean GORDON, San Diego, CA (US); Catalin BARBACIORU, Fremont, CA (US); Evan WARNER, Vancouver (CA); Andrew M. GROSS, Cincinnati, OH (US); Denis TOLKUNOV, Dublin, CA (US); Geethika YALAMANCHILI, Palo Alto, CA (US)

(21) Appl. No.: 19/400,921

(22) Filed: Nov. 25, 2025

Related U.S. Application Data
(60) Provisional application No. 63/724,549, filed on Nov. 25, 2024.

(51) Int. Cl. G16B 25/10; G16B 5/20; G16B 20/20; G16B 30/00; G16H 50/20

(52) U.S. Cl. CPC G16B 25/10; G16B 5/20; G16B 20/20; G16B 30/00; G16H 50/20

(57) ABSTRACT

Methods are provided for detecting gene deletions and driver mutations using tumor-specific methylation patterns from cell-free DNA and tissue biopsies. The methods exploit the mutual exclusivity between tumor-specific methylation and homozygous gene deletion, enabling detection through absence of expected methylation signals. Applications include detection of MTAP, PTEN, and RB1 deletions, as well as prediction of EGFR single nucleotide (SNV) and small (up to 50 base pair) insertions and deletions (indel) driver mutations, enabling identification of patients eligible for targeted therapies.

### CROSS-REFERENCE TO RELATED APPLICATIONS

This application claims priority to U.S. Provisional Patent Application No. 63/724,549, filed Nov. 25, 2024, entitled “Tumor-Specific Methylation-Based Detection of Gene Deletions,” the entire contents of which are incorporated herein by reference.

### FIELD OF THE INVENTION

The present invention relates generally to methods for detecting gene deletions and driver mutations in cancer using tumor-specific methylation patterns from cell-free DNA (cfDNA) and tissue biopsies. More specifically, the invention provides multi-omic approaches that integrate epigenomic and genomic data to detect homozygous deletions, single-copy number losses, and clinically relevant mutations with enhanced sensitivity, particularly in samples with low tumor fraction or low tumor purity.

### BACKGROUND

Cancer genomic profiling has become essential for identifying actionable therapeutic targets and guiding treatment decisions. Detection of gene deletions, particularly homozygous deletions (HomDel), is critical for identifying patients eligible for targeted therapies. For example, methylthioadenosine phosphorylase (MTAP) is a tumor suppressor gene located adjacent to CDKN2A on chromosomal locus 9p21.3. In approximately 15% of all cancers, this locus is deleted, with MTAP co-deleted with CDKN2A in over 80% of cases. Clinical trials investigating PRMT5 and MAT2A inhibitors have demonstrated synthetic lethality in MTAP-deleted tumors, making accurate detection of MTAP HomDel essential for patient selection. Examples include Chen et al., J Immunother Cancer (2024) and Kalev et al., Cancer Cell (2021), each of which is fully incorporated by reference herein.

Conventional methods for detecting gene deletions rely on sequencing coverage analysis, comparing read depth at target loci to reference regions. However, these methods face significant limitations in samples with low tumor fraction (TF), such as liquid biopsies from patients with low tumor shedding, or tissue biopsies with low tumor purity. In mixed samples containing both tumor and non-tumor DNA, the coverage signal from homozygous deletions is diluted by normal DNA, causing sensitivity to drop rapidly as tumor fraction decreases.

DNA methylation patterns are known to differ between tumor and normal cells, with cancer-specific hypermethylation occurring at specific promoter regions. Recent studies have shown that MTAP loss leads to global changes in DNA methylation, with many loci exhibiting reduced tumor-specific hypermethylation in MTAP-deleted tumors. This biological phenomenon creates an opportunity to leverage tumor-specific methylation as a biomarker for gene deletion status.

There remains a need for improved methods to detect gene deletions with high sensitivity and specificity, particularly in low tumor fraction samples where conventional genomic methods fail.

### SUMMARY OF THE INVENTION

Described herein is a method, including: generating a methylation profile for at least one nucleic acid sequence obtained from a human subject, wherein the methylation profile includes at least one differentially menthylated region (DMR). In other embodiments, the at least one DMR is determined based on a comparison to a threshold determined from one or more healthy subjects, optionally including a DMR that is tumor-specific. In other embodiments, the methylation profile characterizes a gain of function for at least one or more genes. In other embodiments, the methylation profile characterizes a loss of function for at least one or more genes. In other embodiments, the methylation profile characterizes a genomic mutation. In other embodiments, the methylation profile characterizes a genetic target, optionally including a genetic variant. In other embodiments, the methylation profile characterizes gene dysfunction. In other embodiments, the characterization is based on hypermethylation status. In other embodiments, the method includes obtaining nucleic acid from a biological sample including cell-free DNA or tissue DNA. In various embodiments, a DMR comprises methylation status and/or methylation levels, of one or loci. In various embodiments, DMRs are determined in comparison to normal healthy subjects, or another disease cohort (e.g., various stages of cancer, or different cancers), which optionally includes comparison to a threshold. In other embodiments, the method includes determining tumor-specific methylation status at a plurality of loci proximal to a genomic mutation, genetic target, optionally including genetic variant. In other embodiments, the method includes determining tumor-specific methylation status at a plurality of loci distal to the genetic target. In other embodiments, the method includes applying a classifier that integrates the proximal and distal methylation features to generate a probability score for homozygous deletion of the target gene, optionally including a trained classifier. In other embodiments, the method includes classifying the sample as having or not having a genomic mutation, genetic variant, gene dysfunction, based on the probability score. In other embodiments, the method includes classifying the sample as having or not having homozygous deletion based on the probability score. In various embodiments, the method includes application of one of more of Equations, 1, 2, 3, 4, 5, 6, 7, and/or 8. In other embodiments, the genetic target is one or more genes selected from the group consisting of MTAP, CDKN2A, PTEN, and RB1. In other embodiments, the biological sample has a tumor fraction of less than 1%, less than 2%, less than 3%, less than 5%, less than 10%. In other embodiments, the biological sample has a tumor fraction of less than 2%. In other embodiments, the trained classifier includes a logistic regression model with elastic net regularization. In other embodiments, the plurality of loci proximal to the target gene includes at least differentially methylated regions within 0.1-0.5, 0.5-1, 2, 3, MB of the target gene. In other embodiments, the genetic target includes MTAP and the plurality of loci proximal to MTAP includes methylated regions adjacent to or within MTAP, CDKN2A, CDKN2A-DT, CDKN2B-AS1, DMRTA1, MIR31HG, and HACD4 genes. In other embodiments, the method includes determining tumor-specific methylation status includes sequencing bisulfite-converted DNA. In other embodiments, the methylation features comprise binary indicators of methylation presence or absence, a probabilistic function or other quantitative measure. In other embodiments, the methylation features comprise quantitative measures of methylation levels.

Additionally described herein is a method for characterizing a biological sample, including: obtaining nucleic acid from a biological sample including cell-free DNA or tissue DNA; determining tumor-specific methylation levels at a plurality of loci proximal to the target gene; determining tumor-specific methylation levels at a plurality of loci distal to the target gene; and applying a classifier to characterize the sample, optionally including a trained classifier. In other embodiments, the method includes detecting single-copy number loss of a target gene in a biological sample, including: obtaining nucleic acid from a biological sample including cell-free DNA or tissue DNA; determining tumor-specific methylation levels at a plurality of loci proximal to the target gene; determining tumor-specific methylation levels at a plurality of loci distal to the target gene; applying a trained classifier that identifies reduction but not complete absence of proximal methylation relative to distal methylation; and characterizing the sample, optionally including single-copy number loss based on the reduction in proximal methylation. In various embodiments, the method includes application of one of more of Equations, 1, 2, 3, 4, 5, 6, 7, and/or 8. weln other embodiments, the target gene is selected from the group consisting of MTAP, PTEN, and RB1. In other embodiments, the method includes applying at least one classifier to the methylation features to generate a methylation-based probability score; applying at least one additional classifier to the other features to generate a genomics-based probability score; integrating the methylation-based and genomics-based probability scores through a weighted ensemble; and characterizing the sample as having or not having a genomic mutation, genetic variant, gene dysfunction, based on the integrated score. In other embodiments, the method includes integrating the methylation-based and genomics-based probability scores through a weighted ensemble. In other embodiments, the weighted ensemble includes: (Equation 8) final=w₁×P_methylation+w₂×P_coverage where w₁ and w₂ are weights determined by cross-validation. In other embodiments, the method includes determining tumor-specific methylation status at a plurality of loci associated with a driver mutation; applying a classifier that identifies methylation patterns indicative of the driver mutation; and predicting presence or absence of the driver mutation based on the methylation patterns, optionally including a trained classifier In other embodiments, the driver mutation includes one or more EGFR mutation is selected from the group consisting of: Exon 19 in-frame deletion, L858R, G719, and L861Q.

Additionally described herein is a method, including: (a) obtaining cell-free DNA from a plasma sample; (b) determining tumor-specific methylation at methylated regions in or adjacent to one or more genes selected from the group consisting of: MTAP, CDKN2A, CDKN2A-DT, CDKN2B-AS1, DMRTA1, MIR31HG, and HACD4; (c) determining tumor-specific methylation at distal control loci; (d) determining genome-wide methylation reduction patterns characteristic of MTAP deletion; (e) applying a logistic regression model integrating the proximal methylation, distal methylation, and genome-wide methylation features; (f) generating a probability score for MTAP homozygous deletion; and (g) classifying the sample, optionally including classifying the sample as as MTAP HomDel positive or negative based on the probability score. In other embodiments, the tumor fractions 5% or lower, 4% or lower, 3% or lower, 2% or lower, and/or 1% or lower. In other embodiments, the method achieves positive predictive value of at least 90% and negative percent agreement of at least 90%. In other embodiments, the method includes integrating the methylation-based probability score with a genomic copy number-based probability score to generate a final integrated score.

Further described herein is a method for identifying patients eligible for PRMT5 or MAT2A inhibitor therapy, including: detecting MTAP homozygous deletion in a patient sample using the method of claim 20; and identifying patients with MTAP homozygous deletion as eligible for PRMT5 or MAT2A inhibitor therapy.

Further described herein is a non-transitory computer-readable medium storing instructions that, when executed by a processor, cause the processor to perform the method of any preceding claim.

Further described herein is a system configured to perform the method of any preceding claim.

Further described herein is a system for detecting gene deletions, including: a sequencing platform configured to generate methylation and genomic data from a biological sample; a processor configured to receive methylation data for loci proximal and distal to a target gene; receive genomic copy number data for the target gene; (apply trained classifiers to generate methylation-based and genomics-based probability scores; integrate the probability scores through a weighted ensemble; and output a classification of gene deletion status, optionally including a display for presenting the classification to a user. In other embodiments, the method detects homozygous deletion with sensitivity that scales with √TF rather than TF, where TF is tumor fraction. In other embodiments, the trained classifier is trained on data from at least 10,000 samples spanning multiple cancer types. In other embodiments, the trained classifier is balanced for tumor fraction and cancer type to avoid bias. In other embodiments, the methylation profile is detected using a methyl binding domain (MBD) partitioning assay. In other embodiments, the MBD partitioning assay includes combining a plurality of nucleic acid molecules derived from the human subject with a solution including an amount of methyl binding domain (MBD) proteins to produce a nucleic acid-MBD protein solution; and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least the threshold cytosine-guanine content. In other embodiments, the treatment includes an PRMT5-MTA5 inhibitor and/or immunotherapy. In other embodiments, the method of any preceding claim, wherein the genomic mutation and/or gene dysfunction modulates PRMT5-MTA complex and/or MTA accumulation.

Described herein is a method, including: detecting methylation in at least one of a plurality of sites; generating a plurality of one or more metrics for each of the plurality of sites; and processing the one or more metrics to characterize at least one cis regulatory network element in a regulatory network. In various embodiment, characterizing the at least one cis regulatory network element includes determine the presence, absence of at least one cis regulatory network element in a regulatory network. In various embodiment, the at least one cis regulatory network element includes, for example, a genetic variant, an indel, a homodel, a fusion, and/or other mutation. In various embodiments, the determination characterizes a sample. In other embodiments, the at least one cis regulatory network element is capable of altering a regulatory sub-circuit. In other embodiments, the cis regulatory network element includes an epigenetic regulator. In other embodiments, the regulatory sub-circuit is bi-directional. In other embodiments, the regulatory sub-circuit increases methylation in plurality of targets. In other embodiments, the regulatory sub-circuit increases methylation in plurality of targets. In other embodiments, the regulatory network includes an additional cis regulatory network element. In other embodiments, the additional cis regulatory network element is capable of altering the regulatory sub-circuit. In other embodiments, the additional cis regulatory network element includes a regulatory node. In other embodiments, the regulatory node is a point of interdiction. In other embodiments, the regulatory sub-circuit includes an additional regulatory node. In other embodiments, the additional regulatory node is a point of interdiction.

Described herein is a method for detecting homozygous deletion of a target gene in a biological sample, including: obtaining nucleic acid from a biological sample including cell-free DNA or tissue DNA; determining tumor-specific methylation status at a plurality of loci proximal to the target gene; determining tumor-specific methylation status at a plurality of loci distal to the target gene; applying a trained classifier that integrates the proximal and distal methylation features to generate a probability score for homozygous deletion of the target gene; and classifying the sample as having or not having homozygous deletion based on the probability score. In other embodiments, the target gene is selected from the group consisting of MTAP, CDKN2A, PTEN, and RB1. In other embodiments, the biological sample has a tumor fraction of less than 5%. In other embodiments, biological sample has a tumor fraction of less than 2%. In other embodiments, the trained classifier is a logistic regression model with elastic net regularization. In other embodiments, the plurality of loci proximal to the target gene includes at least differentially methylated regions within 1 MB of the target gene. In other embodiments, target gene is MTAP and the plurality of loci proximal to MTAP includes methylated regions adjacent to or within MTAP, CDKN2A, CDKN2A-DT, CDKN2B-AS1, DMRTA1, MIR31HG, and HACD4 genes. In other embodiments, determining tumor-specific methylation status includes sequencing bisulfite-converted DNA. In other embodiments, the methylation features comprise binary indicators of methylation presence or absence. In other embodiments, the methylation features comprise quantitative measures of methylation levels.

Further described herein is a method for detecting single-copy number loss of a target gene in a biological sample, including: obtaining nucleic acid from a biological sample including cell-free DNA or tissue DNA; (b) determining tumor-specific methylation levels at a plurality of loci proximal to the target gene; determining tumor-specific methylation levels at a plurality of loci distal to the target gene; applying a trained classifier that identifies reduction but not complete absence of proximal methylation relative to distal methylation; and classifying the sample as having single-copy number loss based on the reduction in proximal methylation. In other embodiments, the target gene is selected from the group consisting of MTAP, PTEN, and RB1.

Further described herein is a multi-omic method for detecting gene deletion, including: (a) obtaining nucleic acid from a biological sample; determining tumor-specific methylation features at loci proximal and distal to a target gene; determining genomic copy number features at the target gene locus; applying a first classifier to the methylation features to generate a methylation-based probability score; applying a second classifier to the copy number features to generate a genomics-based probability score; integrating the methylation-based and genomics-based probability scores through a weighted ensemble; and (g) classifying the sample as having or not having gene deletion based on the integrated score. In other embodiments, the weighted ensemble includes: P_final=w₁×P_methylation+w₂×P_coverage where w₁ and w₂ are weights determined by cross-validation. In other embodiments, the multi-omic method achieves at least 2-fold improvement in sensitivity compared to genomics-based methods alone.

Further described herein is a method for detecting gene deletion in a tissue biopsy with low tumor purity, including: (a) obtaining nucleic acid from a tissue biopsy sample having tumor purity less than 20%; (b) determining tumor-specific methylation status at a plurality of loci proximal to a target gene; (c) determining tumor-specific methylation status at a plurality of loci distal to the target gene; (d) applying a trained classifier to generate a probability score for gene deletion; and (e) detecting gene deletion that would be undetectable by conventional coverage-based methods due to the low tumor purity.

In other embodiments, the tissue biopsy sample has tumor purity less than 10%.

Further described herein is a method for predicting presence of a driver mutation in a biological sample, including: obtaining nucleic acid from a biological sample including cell-free DNA or tissue DNA; determining tumor-specific methylation status at a plurality of loci associated with the driver mutation; applying a trained classifier that identifies methylation patterns indicative of the driver mutation; and predicting presence or absence of the driver mutation based on the methylation patterns. In other embodiments, the driver mutation is an EGFR mutation selected from the group consisting of Exon 19 in-frame deletion, L858R, G719, and L861Q.

Further described herein is a method for detecting MTAP homozygous deletion with enhanced sensitivity, including: (a) obtaining cell-free DNA from a plasma sample; (b) determining tumor-specific methylation at methylated regions in or adjacent to MTAP, CDKN2A, CDKN2A-DT, CDKN2B-AS1, DMRTA1, MIR31HG, and HACD4; (c) determining tumor-specific methylation at distal control loci; (d) determining genome-wide methylation reduction patterns characteristic of MTAP deletion; (e) applying a logistic regression model integrating the proximal methylation, distal methylation, and genome-wide methylation features; (f) generating a probability score for MTAP homozygous deletion; and (g) classifying the sample as MTAP HomDel positive or negative based on the probability score. In other embodiments, the method achieves detection at tumor fractions as low as 1%. In other embodiments, the method achieves positive predictive value of at least 90% and negative percent agreement of at least 90%.

In other embodiments, the method further includes integrating the methylation-based probability score with a genomic copy number-based probability score to generate a final integrated score.

Additionally described herein is a method for identifying patients eligible for PRMT5 or MAT2A inhibitor therapy, including: (a) detecting MTAP homozygous deletion in a patient sample using the method of claim 20; and (b) identifying patients with MTAP homozygous deletion as eligible for PRMT5 or MAT2A inhibitor therapy.

Additionally described herein is a system for detecting gene deletions, including: a sequencing platform configured to generate methylation and genomic data from a biological sample; a processor configured to: receive methylation data for loci proximal and distal to a target gene; receive genomic copy number data for the target gene; apply trained classifiers to generate methylation-based and genomics-based probability scores; integrate the probability scores through a weighted ensemble; and output a classification of gene deletion status; and a display for presenting the classification to a user.

Additionally described herein is a non-transitory computer-readable medium storing instructions that, when executed by a processor, cause the processor to perform the method of any one of claims 1, 11, 13, 16, 18, or 20.

Additionally described herein is a kit for detecting gene deletions, including: (a) reagents for bisulfite conversion of DNA; (b) primers or probes for amplifying or detecting methylation status at loci proximal and distal to a target gene; (c) reagents for sequencing or methylation analysis; and (d) instructions for performing the method of claim 1.

In other embodiments, the method detects homozygous deletion with sensitivity that scales with √TF rather than TF, where TF is tumor fraction. In other embodiments, the trained classifier is trained on data from at least 10,000 samples spanning multiple cancer types. In other embodiments, the trained classifier is balanced for tumor fraction and cancer type to avoid bias.

The present invention provides novel methods for detecting gene deletions (both homozygous deletions and single-copy number losses) and driver mutations using tumor-specific methylation patterns from cell-free DNA and tissue biopsies. The invention is based on the discovery that tumor-specific methylation at a genomic locus is mutually exclusive with homozygous deletion of that same locus, enabling detection of deletions through absence of expected methylation signals.

In one aspect, the invention provides a method for detecting homozygous deletion of a target gene in a biological sample, including: (a) obtaining nucleic acid from a biological sample including cell-free DNA or tissue DNA; (b) determining tumor-specific methylation status at a plurality of loci proximal to the target gene; (c) determining tumor-specific methylation status at a plurality of loci distal to the target gene; (d) applying a trained classifier that integrates the proximal and distal methylation features to generate a probability score for homozygous deletion of the target gene; and (e) classifying the sample as having or not having homozygous deletion based on the probability score.

In another aspect, the invention provides a method for detecting single-copy number loss of a target gene, wherein reduction but not complete absence of tumor-specific methylation indicates loss of one gene copy.

In yet another aspect, the invention provides a multi-omic method that integrates tumor-specific methylation features with conventional genomic copy number analysis to improve both sensitivity and specificity of deletion detection.

In a further aspect, the invention provides methods for detecting gene deletions in tissue biopsies with low tumor cellularity or purity, where conventional coverage-based methods fail due to insufficient tumor DNA molecules.

In still another aspect, the invention provides methods for predicting the presence of clinically relevant single nucleotide variants (SNVs) and insertions/deletions (indels) using tumor-specific methylation patterns, including EGFR driver mutations such as Exon 19 in-frame deletions, L858R, G719, and L861Q variants.

The invention demonstrates superior performance compared to conventional methods, with area under the curve (AUC) values of 0.93 for MTAP HomDel detection, positive predictive value (PPV) of 100%, and negative percent agreement (NPA) of 100% above the limit of detection. The multi-omic methodology achieves over 2.4-fold improvement in sensitivity compared to genomic-only methods.

Described herein is a method, including: generating a methylation profile for at least one nucleic acid sequence obtained from a human subject; and selecting a treatment suitable for the human subject based on the methylation profile. In other embodiments, the methylation profile includes at least one differentially menthylated region (DMR) In other embodiments, the at least one DMR is determined based on a comparison to a threshold determined from one or more healthy subjects. In other embodiments, the methylation profile characterizes a gain of function for at least one or more genes. In other embodiments, the methylation profile characterizes a loss of function for at least one or more genes. In other embodiments, the methylation profile characterizes a genomic mutation. In other embodiments, the methylation profile characterizes a gene target. In other embodiments, the methylation profile characterizes gene dysfunction. In other embodiments, the characterization is of a gene target capable of conferring synthetic lethality. In other embodiments, the characterization is based on hypermethylation status. In other embodiments, the one or more genes includes CDKN2A. In other embodiments, the one or more genes includes a metabolic enzyme. In other embodiments, the metabolic enzyme includes Methylthioadenosine Phosphorylase (MTAP). In other embodiments, the methylation profile is detected using a methyl binding domain (MBD) partitioning assay. In other embodiments, the MBD partitioning assay includes combining a plurality of nucleic acid molecules derived from the human subject with a solution including an amount of methyl binding domain (MBD) proteins to produce a nucleic acid-MBD protein solution; and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least the threshold cytosine-guanine content. In other embodiments, the treatment includes an PRMT5-MTA5 inhibitor and/or immunotherapy. In other embodiments, the genomic mutation and/or gene dysfunction modulates PRMT5-MTA complex and/or MTA accumulation. In other embodiments, the e detection includes a method selected from the group consisting of Next Generation Sequencing (NGS), Third-Generation Sequencing (TGS), Sanger Sequencing, Microarray Analysis, PCR, Fluorescence In Situ Hybridization (FISH), Real-Time PCR (RT-PCR), Digital PCR (dPCR), Droplet Digital PCR (ddPCR), Targeted Sequencing, Nanopore Sequencing, Short-Read Sequencing, Long-Read Sequencing Technologies (e.g., PacBio and Nanopore). In other embodiments, the e patient is afflicted with a cancer selected form the group consisting of: breast cancer, lung cancer, prostate cancer, colorectal cancer, skin cancer, ovarian cancer, pancreatic cancer, leukemia, lymphoma, liver cancer, cervical cancer, brain cancer, stomach cancer, kidney cancer, bladder cancer, thyroid cancer, esophageal cancer, melanoma, testicular cancer, epithelioid sarcoma, relapsed or refractory follicular lymphoma, non-Hodgkin lymphomas (NHL), relapsed or refractory adult T-cell leukemia/lymphoma (R/R ATL), follicular lymphoma, castration-resistant prostate cancer (CRPC), small cell lung cancer, and sarcoma. In other embodiments, the sample is selected from the group consisting of blood, plasma, cell free DNA (cfDNA), cell free RNA (cfRNA), saliva, urine, cerebrospinal fluid, synovial fluid, amniotic fluid, lymph, semen, vaginal fluid, tears, breast milk, mucus, sweat, pericardial fluid, pleural fluid, peritoneal fluid, bile, and interstitial fluid. In other embodiments, the method includes selecting a treatment suitable for the human subject includes use of a database. In other embodiments, the database includes a plurality of nucleic acid sequence information, including methylation status from a plurality of subjects. In other embodiments, selecting a treatment includes identifying from a plurality of subjects with matching genetic, epigenetic information, prior treatment of the plurality of subjects with matching genetic information.

Described herein is a method, including: generating a methylation profile using a methyl binding domain (MBD) partitioning assay for at least one nucleic acid sequence obtained from a human subject; selecting a treatment suitable for the human subject based on the methylation profile using a database, wherein the database includes a plurality of nucleic acid sequence information, and methylation status from a plurality of subjects and identifying from the plurality of subjects with matching genetic and epigenetic information, prior treatment of the plurality of subjects with matching genetic information.

### BRIEF DESCRIPTION OF THE FIGURES

FIG. 1. Using >>10% TF for detection of deletions in liquid limits evaluable proportion of samples to <<20%.

FIG. 2. Methylation-based deletion calling uses coverage information from cancer-specific methylated molecules only

FIG. 3. Absence of cancer-specific methylated molecules on and flanking the deleted gene but present elsewhere indicates deletion event. Additional changes in methylation due to loss of biological function of deleted gene.

FIG. 4. Evaluating methylation patterns in selected 127 promoter subset. The above plots show the results for OR≤0.2, min_freq≥10%, for which 127/1494 promoters pass these thresholds. 89% of MTAP homdel samples have 10% or less of these promoters methylated. 73% of MTAP intact samples have more than 10% of these promoters methylated.

FIG. 5. MTAP flanking hyper methylation is tumor-specific and increases with TF

FIG. 6. Promoter hypermethylation has dependence on TF. Summary of MRD (TF=0) and LDT (TF by bin) CDKN2A, CDKN2B-AS1, or CDKN2A-DT promoter methylation frequency observe relative higher frequencies when allowing for methylation of any of three genes as compared to an individual gene promoter

FIG. 7. Deep deletions at the 9p21.3 locus are among most frequent across tumors and co-delete MTAP with CDKN2A at high rate.

FIG. 8. If CDKN2A is deleted then MTAP is extremely likely to also be deleted in the same CN loss state. Homozygous deletions frequently co-occur in MTAP, CDKN2A, and IFN given their close proximity on chromosome 9. samples >10% TF (n=3249)

FIG. 9. MTAP flanking region methylation is tumor-specific and increases with TF. MRD data contains 17609 samples, 2764 of which are tumor positive and 555 are TF>1%.

FIG. 10. Unlike deletion detection, promoter methylation detection has a low LoD near SNV and Indel level. Promoter hypermethylation calls are already representatively validated. One can adjusted thresholds for promoter hypermethylation calls in the context of the described model inputs.

FIG. 11. Hypermethylation of MTAP flanking promoters is linked to both TF and deletion status. Summary of MRD (TF=0) and LDT (TF by bin) CDKN2A promoter methylation frequency

FIG. 12. Promoters close to MTAP show decreased hypermethylation in samples with MTAP homdel, with MTAP LoH intermediate

FIG. 13. Impact of MTAP homdel on promoter methylation decreases in proportion to distance from MTAP gene

FIG. 14. Reduced methylation of MTAP flanking region in MTAP homdel works across different cancer types. Decreased CDKN2A promoter methylation observed in five most prevalent cancer types in LDT cohort Consistently differentiates LoH from Homdel

FIG. 15. Using a combination of 6 different flanking genes yields higher fraction of MTAP-intact models with regional hypermethylation. Use of a single region (e.g., CDKN2A) in isolation leaves a large fraction of samples with no methylation (i.e., apparent MTAP-HD phenotype). Summary of MRD (TF=0) and LDT (TF by bin) promoter methylation frequency for the following genes: CDKN2A, CDKN2A-DT, CDKN2B-AS1, DMRTA1, MIR31HG, HACD4. ^{~}85% of all samples with MTAP intact and TF>10% have evidence of PM in one or more of the above listed genes. ^{~}11% of samples with MTAP homdel have evidence of PM (TF>10%) in one of these flanking regions

FIG. 16. Molecule counts for gene adjacent to MTAP are much lower in MTAP CN loss background regardless of TF.

FIG. 17. Molecule counts for gene adjacent to MTAP are much lower in MTAP CN loss background regardless of TF. Difference between MTAP categories plotted by TF bin

FIG. 18. Prevalence in Tissue is comparable to liquid data Snapshot of CDKN2A promoter methylation frequency tissue.

FIG. 19. Strong difference in proportion of samples hypermethylated between MTAP status groups (10 to 20% TF here).

FIG. 20. Strong difference in number hypermethylated molecules between MTAP groups

FIG. 21. Sum of molecules across samples with respective MTAP status

FIG. 22. illustrates the mutual exclusivity of tumor-specific methylation and homozygous deletion, showing that in the absence of gene copies, the methylation signal at the locus disappears completely.

FIG. 23. Schematic comparison of conventional genomic method versus novel tumor-specific methylation method for deletion detection at low tumor fraction. Left panel shows conventional genomic coverage-based method where non-tumor background DNA (gray) dominates the signal at low tumor fraction (2.4%), masking the deletion present in tumor DNA (red). The coverage graph shows only a subtle dip at the MTAP locus, insufficient for confident deletion calling. Right panel shows the novel tumor-specific methylation method where only tumor DNA contributes to the signal. Tumor-specific methylation (green markers) is present at distal loci but completely absent at the deleted MTAP locus, creating a clear deletion signature despite the low tumor fraction. This approach enables confident MTAP HomDel detection where conventional methods fail. Importantly, here, it is noted that effect size scales linearly with TF. The z-score (and thus detection power) scales linearly with tumor fraction. Further, as described herein normalized effect size remains constant regardless of TFTFTF (comparing presence vs. absence of methylation in tumor cells). The z-score (and detection power) scales with the square root of tumor fraction.

FIG. 24A. depicts mathematical models showing z-score behavior for conventional versus tumor-specific coverage across tumor fractions, demonstrating the more gradual sensitivity decay of tumor-specific methods. FIG. 24B. Why conventional dips fast: In mixed cfDNA, a tumor homdel only reduces total coverage by TF of baseline (2→2·(1−TF)), so the signal vanishes linearly with TF. Why tumor-specific stays linear/robust: By isolating tumor-only molecules, the effect size stays ^{~}constant (2→0) and only the noise grows as tumor molecules get rarer. Sensitivity therefore scales as √TF, not TF. Net result: At low TF, tumor-specific coverage shows a gentle slope (more linear, less cliff-like) in detection vs TF, whereas conventional total-coverage shows a steep drop-off. In real datasets, process noise and thresholding mean the 95% LoDs can be only modestly different, but the operating curve (sensitivity over TF) is clearly superior for the tumor-specific approach.

FIG. 25. ROC curve of methylation-based detection in validation cohort. shows a receiver operating characteristic (ROC) curve demonstrating the accuracy of methylation-enhanced MTAP HomDel detection with AUC of 0.93. Further shown is high probability of methylation-enhanced deletion detection in samples detected for MTAP HomDel by genomic-based detection vs low probability in samples with putative intact or single copy deletion.

FIG. 26. Upper plot illustrates that when including tissue biopsy samples with less than 20% tumor cellularity, detection rate is higher when including tumor specific methylation method for detection of MTAP HomDel relative to MTAP detection by genomic method (orange bar). Bottom plot illustrates that at tumor cellularity less than 20% the application of tumor-specific methylation method (blue) is more sensitive than conventional genomic method (orange). Number of samples per cancer type for this experiment is shown in the upper table and number of samples used by their tumor cellularity is shown in the second table.

FIG. 27. presents comparative performance of the integrated multi-omic classifier at different sensitivity/specificity tradeoffs versus genomic-only methods, demonstrating over 2.4-fold improvement in sensitivity (2.4 fold increase via the highest specificity configuration, shown in green bar; conventional genomic method shown in orange; literature detection rate via tissue shown in blue). Figure uses 56,917 consecutive samples from testing of advanced stage patients for comprehensive genotyping in LDT.

FIG. 28. shows the number of tumor specific methylation loci used (32 loci for RB1; 16 loci for PTEN) and corresponding performance used for RB1 (upper plot) and PTEN (lower plot) methylation-based deletion detection, respectively. Tumor specific methylation method is able to achieve up to 90% sensitivity and 90% specificity for RB1 HomDel detection and 80% sensitivity and 90% specificity for PTEN HomDel detection.

FIG. 29. visualizes methylation reduction patterns in MTAP-deleted tumors across the genome, showing the biological basis for genome-wide methylation features. Top plots show that most frequently there are more loci that are reduced in methylation in MTAP deleted samples (blue points) as compared to loci with increased methylation in MTAP deleted samples. Workflow shows a summary of an analysis to identify specific loci that are either more frequently methylated in MTAP deleted samples (256 loci) versus loci that are more frequently methylated in samples without MTAP deletion (MTAP intact). Bottom plot shows that the final selected loci (127) are useful for the diagnostic purpose of identifying MTAP deleted samples. 89% of MTAP HomDel samples have less than 10% of these selected loci methylated whereas more that 73% of MTAP intact samples have more than 10% of these loci methylated.

FIG. 30. presents a validation study cohort for MTAP HomDel. Analytical accuracy was assessed in 87 matched cfDNA and tissue samples (NSCLC=40%, pancreatic=9%, melanoma=9%, breast=7%, other=35%) from patients with advanced cancer. MTAP HomDel and single copy number loss were treated as distinct categories. Results show NPA and PPV against tissue reference, demonstrating 100% PPV and 100% NPA above limit of detection. The described multi-omic methodology achieved a positive predictive value (PPV) of 100% and negative percent agreement (NPA) of 100% above LoD (90.9% and 96.6% overall) against a tissue-based reference. No false positives were observed among 120 cancer-free donors. Assessment of cfDNA samples from patients with advanced cancer demonstrated that multi-omic MTAP HomDel detection was over 2.4 fold as sensitive as genomic-only methods.

FIG. 31. illustrates application of methylation-based models to EGFR SNV detection from tissue biopsy using TCGA 450K array data, demonstrating extensibility beyond deletion detection. Sensitivity of EGFR driver mutation detection was 84% with a specificity of 87.5%.

FIG. 32. Upper plot illustrates building methylation-based models to predict PTEN HomDel events from tissue biopsy based TCGA 450K array data using 250 selected methylation loci. Lower plot shows performance for RB1 methylation-based models to predict RB1 HomDel events from tissue biopsy based TCGA 450K array data.

FIG. 33. illustrates application of methylation-based models to RB1 detection.

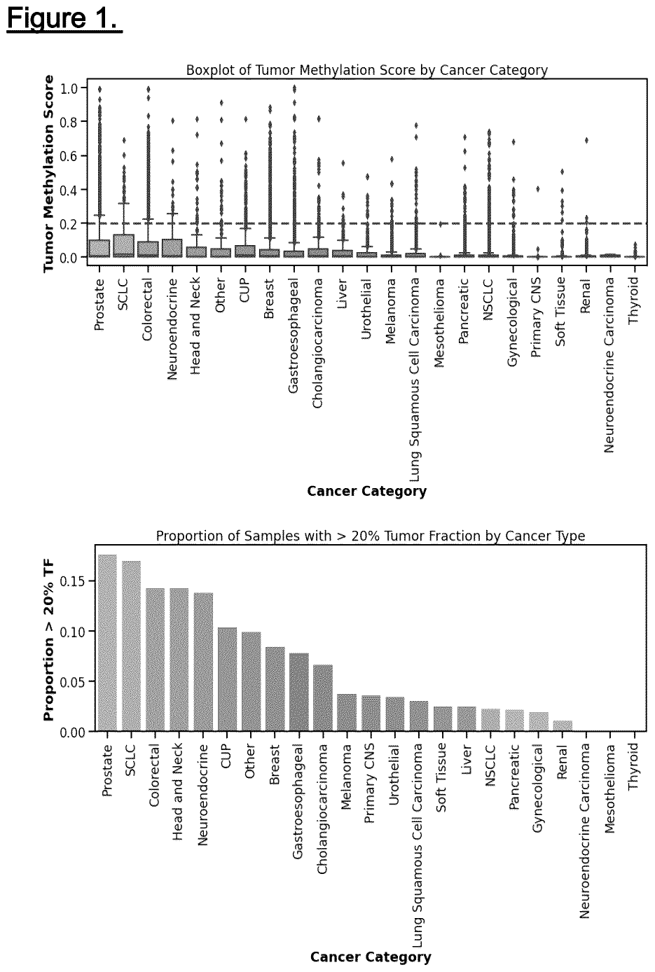

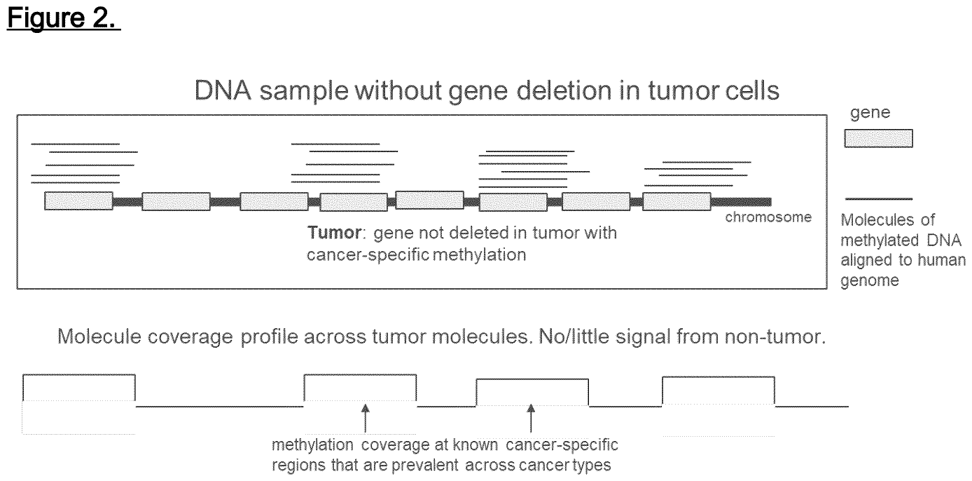

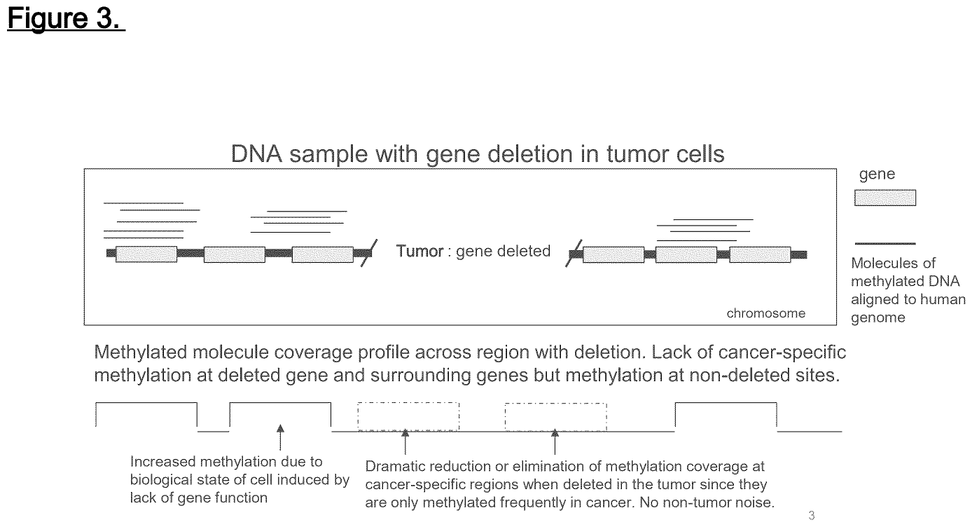

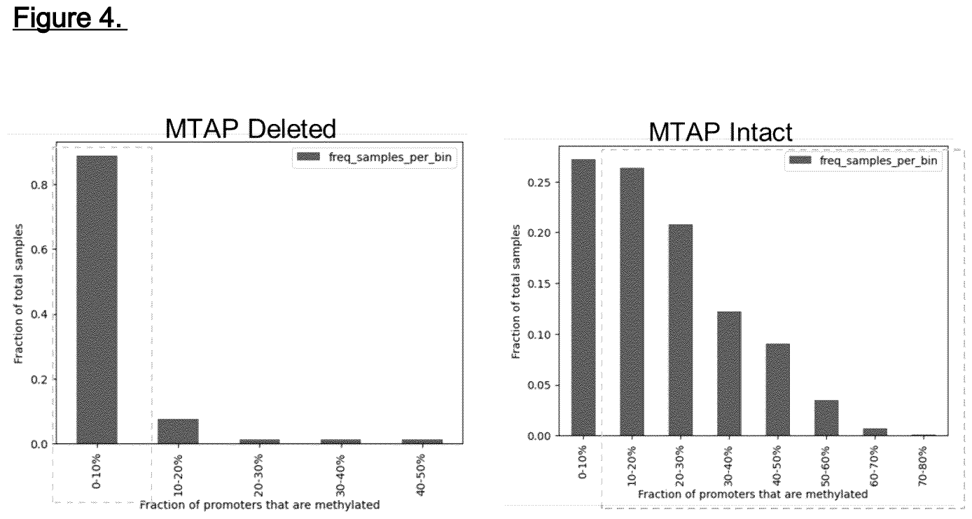

### DETAILED DESCRIPTION OF THE INVENTION

The present invention provides novel methods for detecting gene deletions (both homozygous deletions and single-copy number losses) and driver mutations using tumor-specific methylation patterns from cell-free DNA and tissue biopsies. Without being bound by any particular theory, the method and compositions described herein utilize the fact that tumor-specific methylation at a genomic locus exhibits a degree of exclusivity, including is mutually exclusivity, with homozygous deletion of that same locus, enabling detection of deletions through absence of expected methylation signals. As described, methylthioadenosine phosphorylase (MTAP) is a tumor suppressor involved in the methionine salvage cycle. The MTAP gene is adjacent to the tumor suppressor CDKN2A on the chromosomal locus, 9p21.3. In ^{~}15% of all cancers, the locus 9p21.3 is deleted, and MTAP is co-deleted with homozygous loss of CDKN2A in >80% of cases. Clinical trials investigating PRMT5 and MAT2A inhibitors have demonstrated potential for inducing synthetic lethality in MTAP-deleted tumors. Thus, the characterization of MTAP deletion in solid tumors is necessary to identify eligible patients. However, robust detection of biallelic gene deletion in liquid biopsies requires high levels of tumor shedding. Described herein is the use of methylation detection, including methylome profiling to identify underlying genetic variants and genomic mutations. One example includes performance of a methylation-informed MTAP deletion classifier, which further has capability to resolve copy loss down to 1% tumor fraction. More information is found in PCT App. No. PCT/US2024/050063, which is fully incorporated by reference herein.

As described herein, the methods and compositions provide numerous advantages over conventional genomic methods:
Enhanced Sensitivity at Low Tumor Fraction: The tumor-specific methylation approach maintains detection capability at tumor fractions as low as 1%, where conventional methods fail.
Mathematical Superiority: Signal-to-noise ratio scales with √TF rather than TF, providing more gradual sensitivity decay.
Multi-Modal Integration: Combining methylation and genomic features improves both sensitivity and specificity beyond either modality alone.
Applicability to Multiple Sample Types: The methodology works for both cfDNA and tissue biopsies, including low-purity tissue samples.
Detection of Multiple Deletion Types: Distinguishes between homozygous deletion and single-copy loss with high accuracy.
Extensibility to Multiple Genes: Demonstrated success for MTAP, PTEN, RB1, and potential for other clinically relevant genes.
Driver Mutation Detection: Extends beyond deletions to predict presence of SNVs and indels such as EGFR driver mutations.
High Analytical Performance: PPV of 100% and NPA of 100% above LoD in validation studies.

### Samples

A sample can be any biological sample isolated from a subject. A sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids. A sample can be isolated or obtained from a subject and transported to a site of sample analysis. The sample may be preserved and shipped at a desirable temperature, e.g., room temperature, 4 °C, −20 °C, and/or −80 °C. A sample can be isolated or obtained from a subject at the site of the sample analysis. The subject can be a human, a mammal, an animal, a companion animal, a service animal, or a pet. The subject may have a cancer. The subject may not have cancer or a detectable cancer symptom. The subject may have been treated with one or more cancer therapy, e.g., any one or more of chemotherapies, antibodies, vaccines or biologics. The subject may be in remission. The subject may or may not be diagnosed as being susceptible to cancer or any cancer-associated genetic mutations/disorders.

The volume of plasma can depend on the desired read depth for sequenced regions.

Exemplary volumes are 0.4-40 ml, 5-20 ml, 10-20 ml. For example, the volume can be 0.5 mL, 1 mL, 5 mL 10 mL, 20 mL, 30 mL, or 40 mL. The volume of sampled plasma may be 5 to 20 mL.

A sample can comprise various amounts of nucleic acid that contains genome equivalents. For example, a sample of about 30 ng DNA can contain about 10,000(104) haploid human genome equivalents and, in the case of cfDNA, about 200 billion (2×1011) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant (i.e. a chemical or protein modification), wherein the epigenetic variant is associated with the presence of a genetic variant such as a cancer-associated mutation. In some embodiments, the sample includes an epigenetic variant associated with the presence of a genetic variant, wherein the sample does not comprise the genetic variant.

Exemplary amounts of cell-free nucleic acids in a sample before amplification range from about 1 fg to about 1 μg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng.

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides. Cell-free nucleic acids can be isolated from bodily fluids through a fractionation or partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with alcohol. Further clean up steps may be used such as silica based columns to remove contaminants or salts. Non-specific bulk carrier nucleic acids, such as Cot-1 DNA, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

### Application to Tissue Biopsies

The tumor-specific methylation methodology is transferable to tissue-based assays. This includes tissue informed testing for tumor subtyping, minimal residual disease, recurrence, etc. tissue assay. In various embodiments, tissue informed tests takes tissue biopsy as input and includes enrichment for broad methylation coverage. It is readily appreciated that the aforementioned methods are applicable to the same principles to tissue samples.

Analytical accuracy was assessed using 87 matched cfDNA and tissue samples from patients with advanced cancer, including NSCLC (40%), pancreatic (9%), melanoma (9%), breast (7%), and other cancer types (35%). MTAP HomDel and single-copy number loss were treated as distinct categories.

The multi-omic methodology achieved positive predictive value (PPV) of 100% and negative percent agreement (NPA) of 100% above the limit of detection (90.9% and 96.6% overall) against a tissue-based reference.

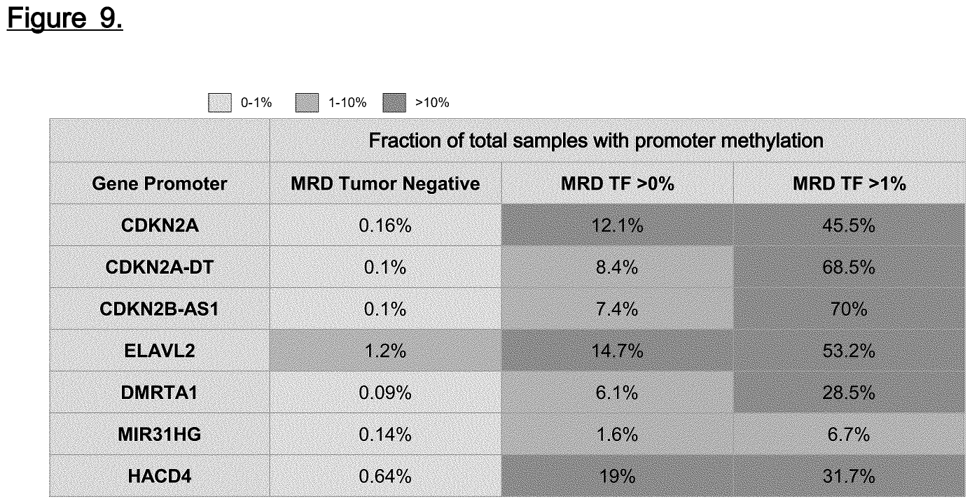

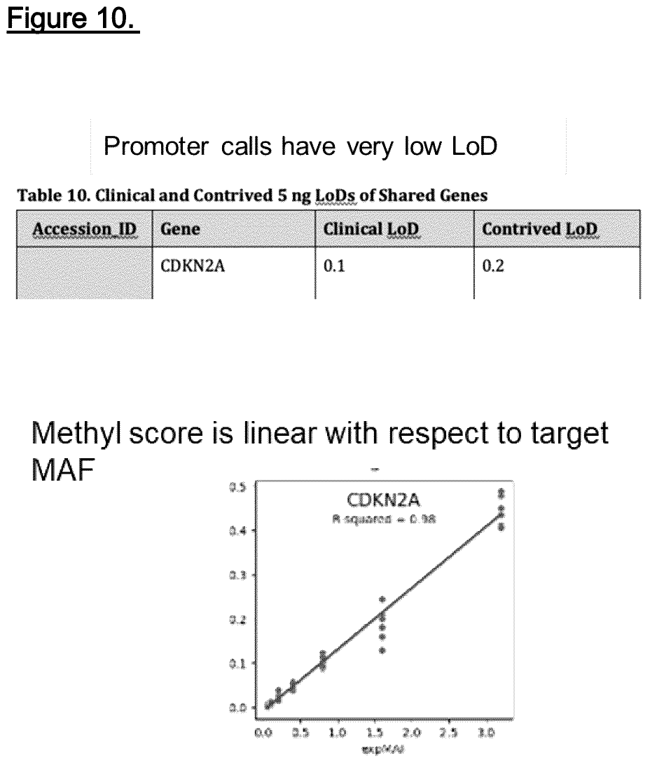

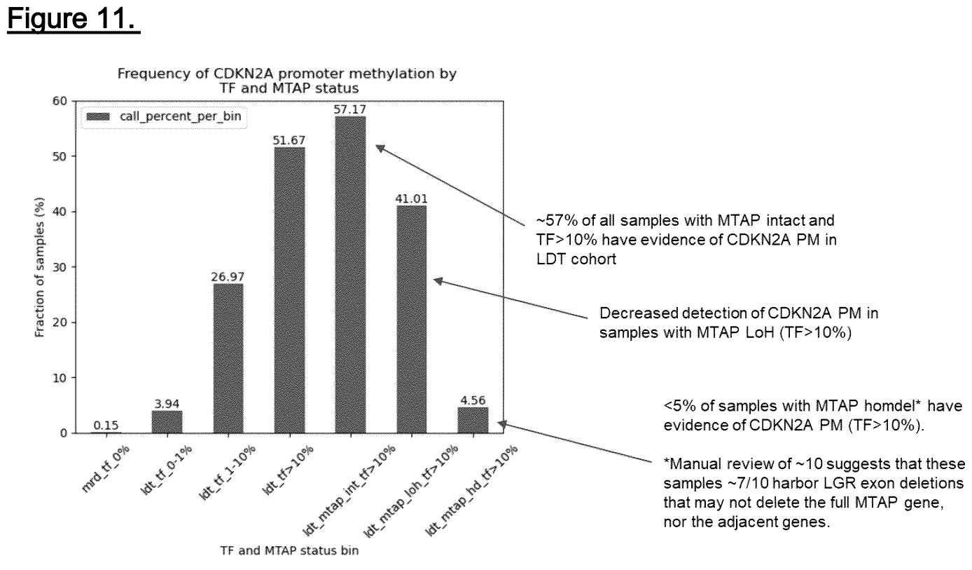

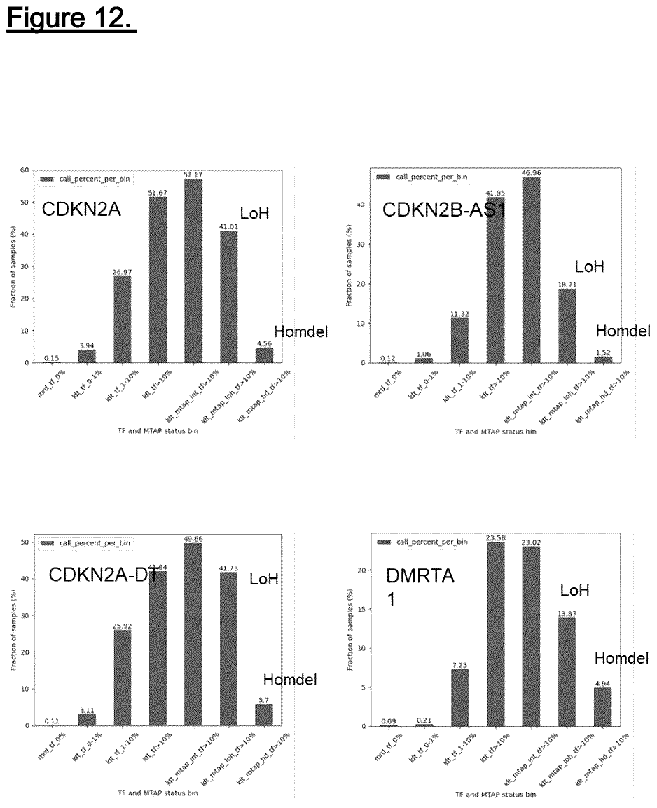

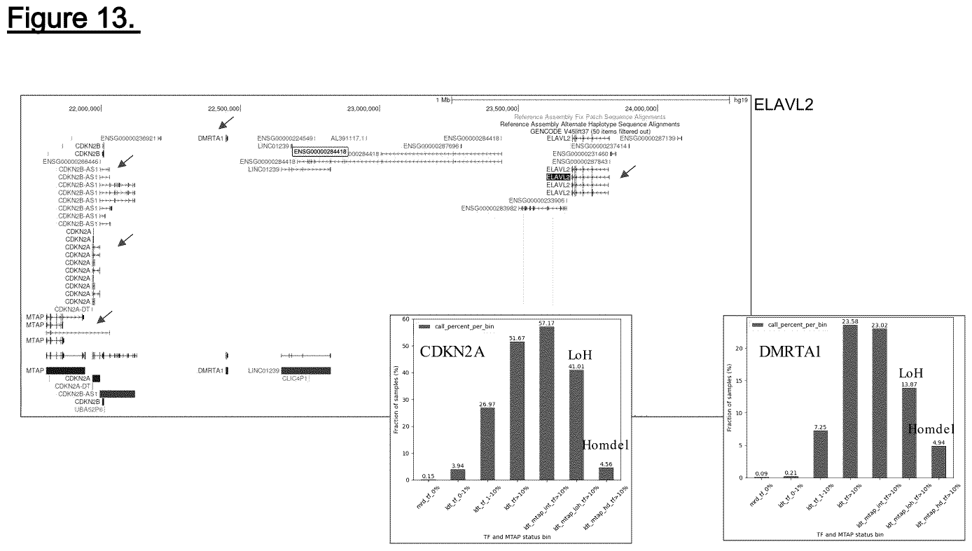

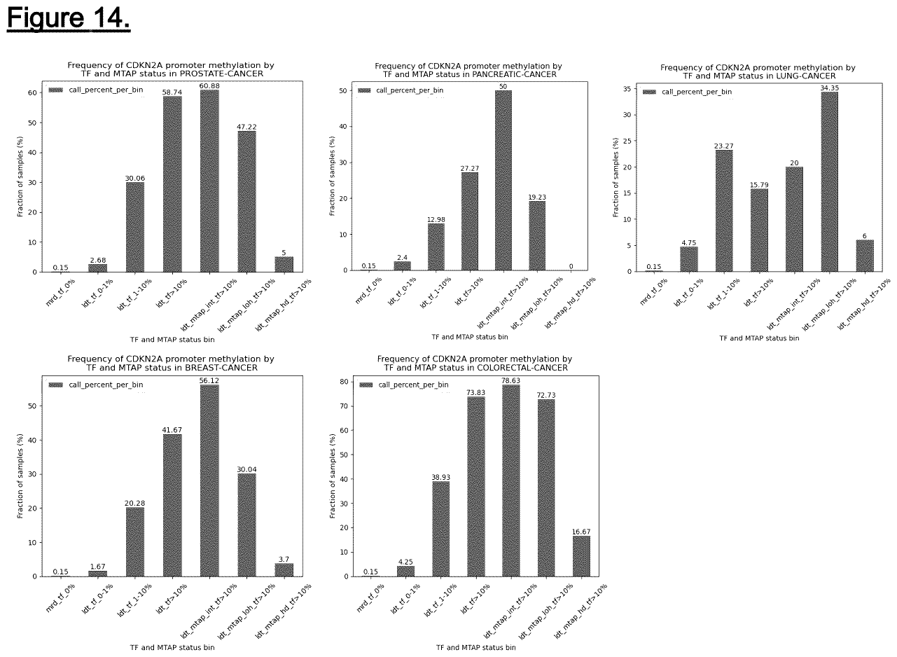

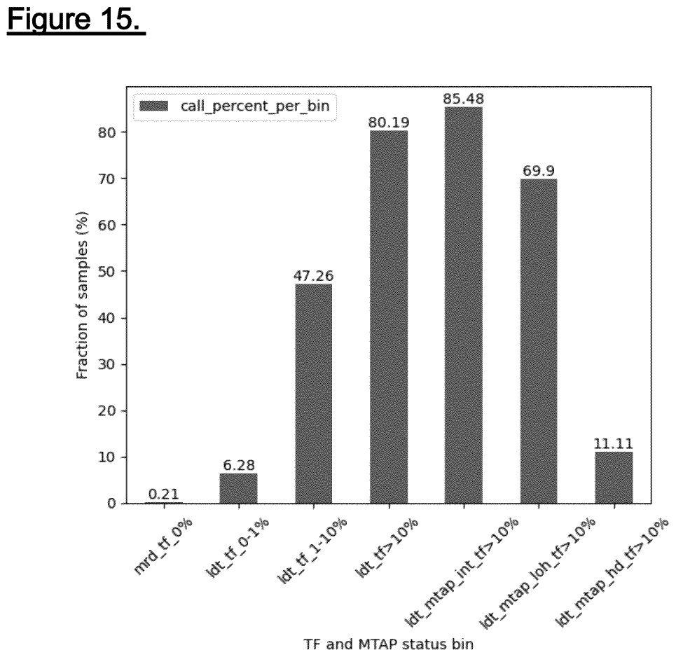

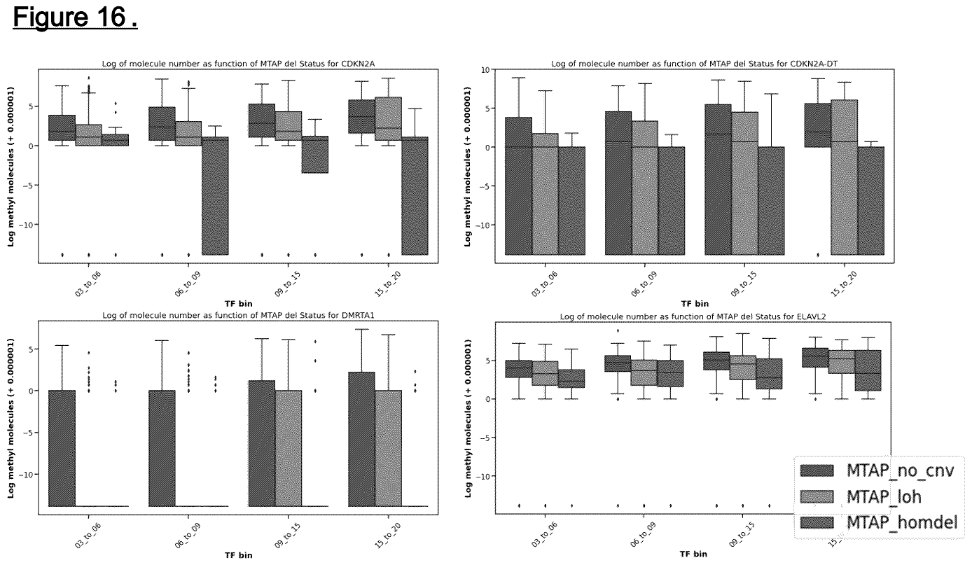

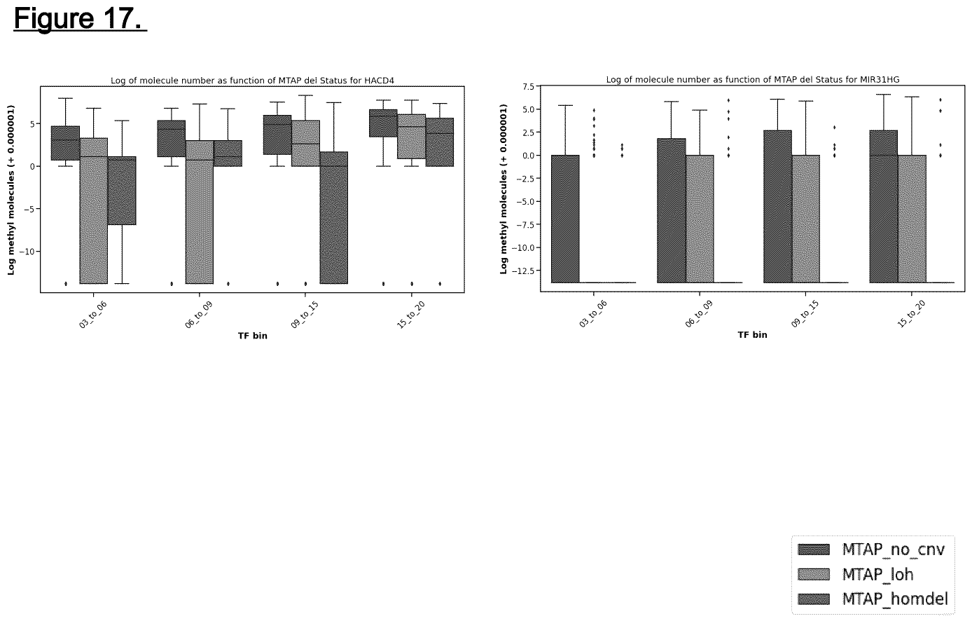

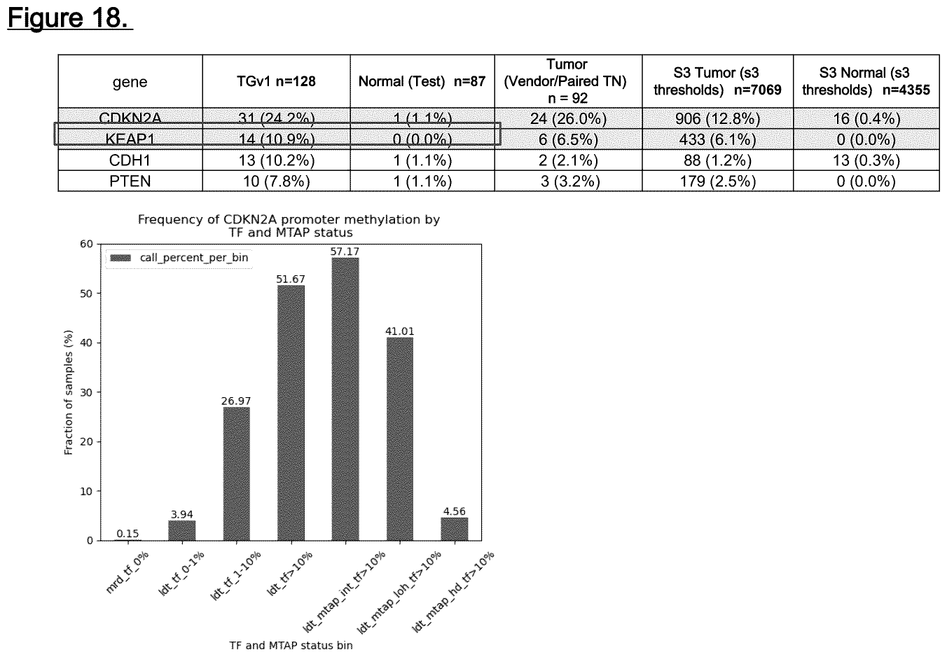

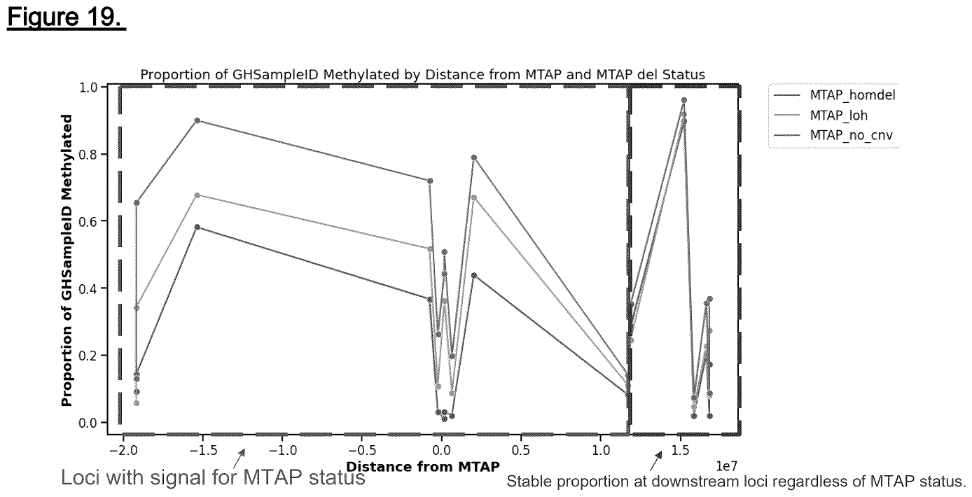

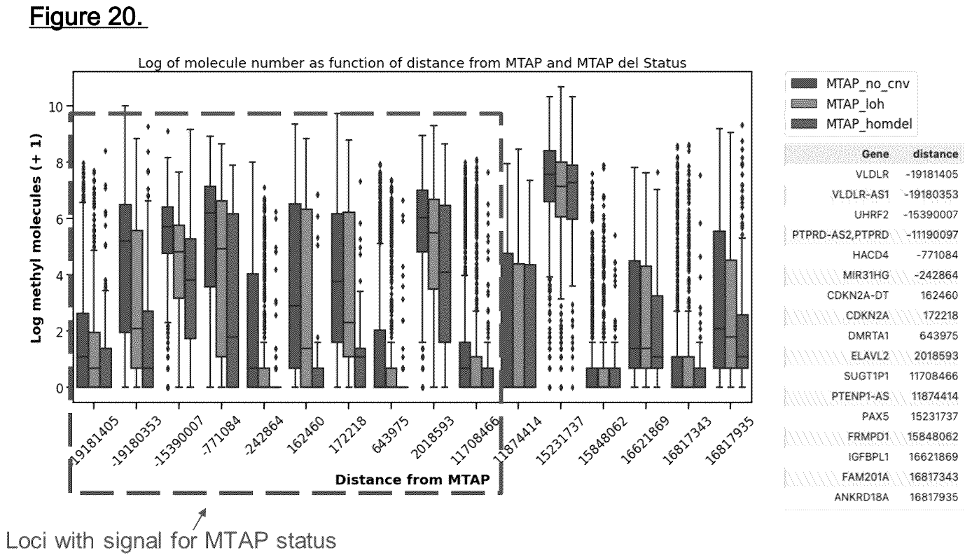

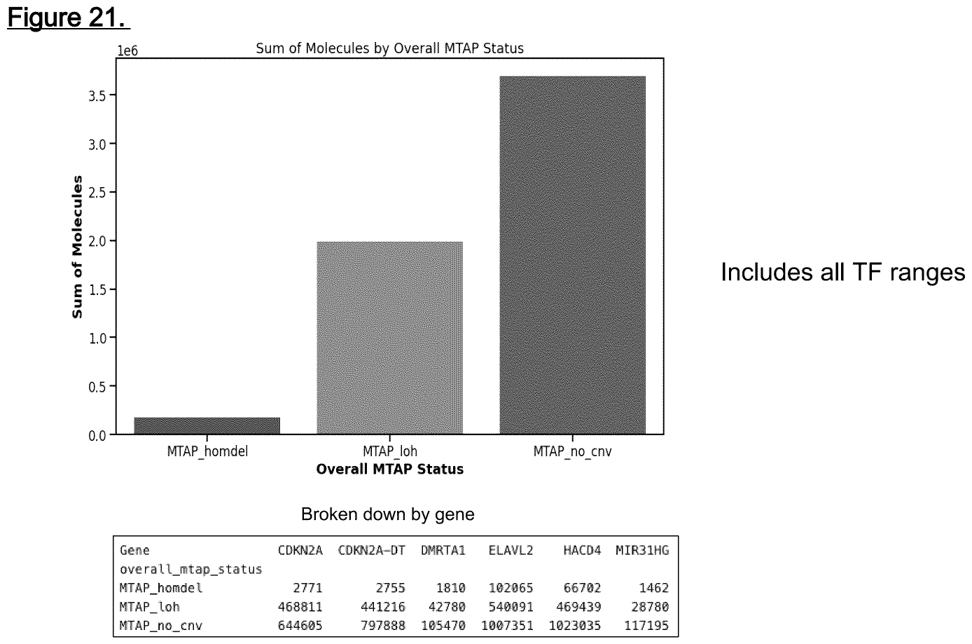

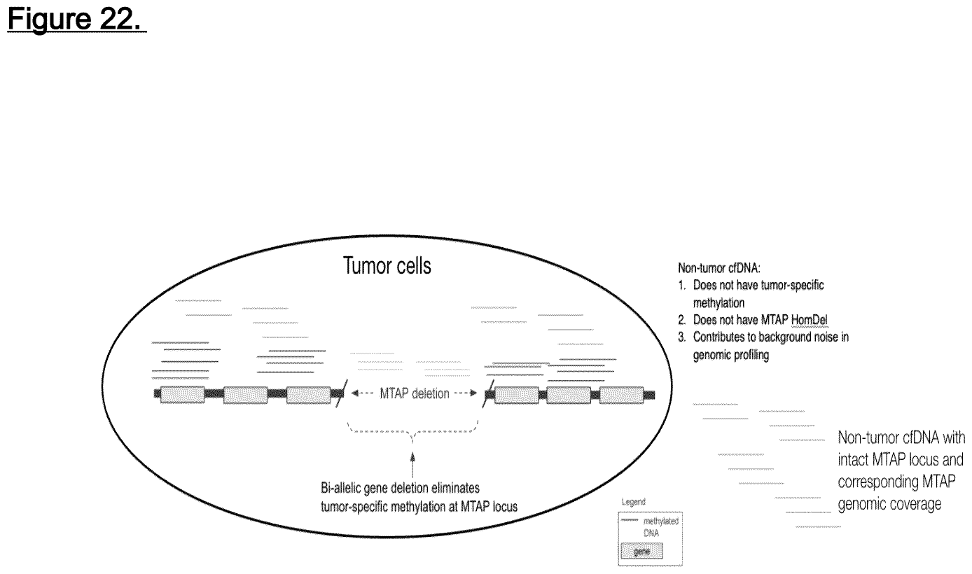

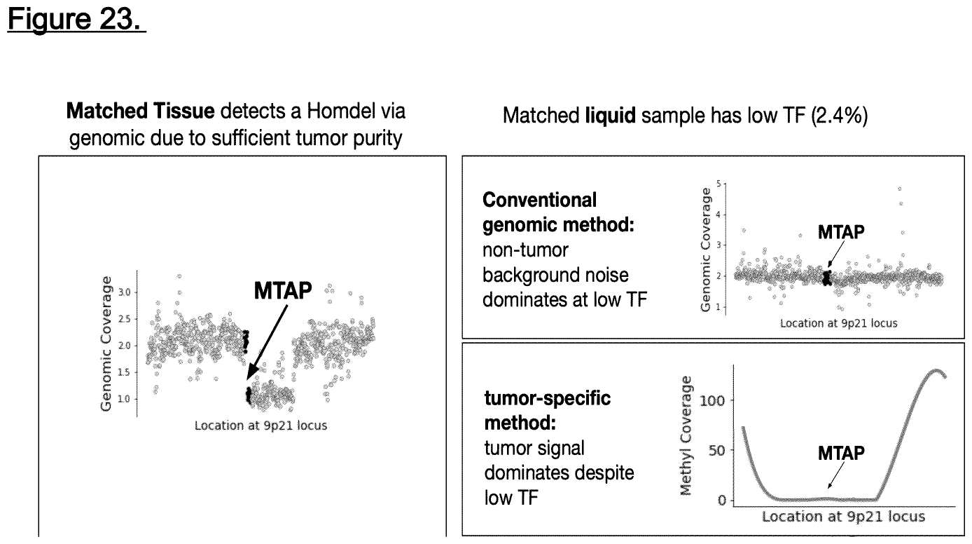

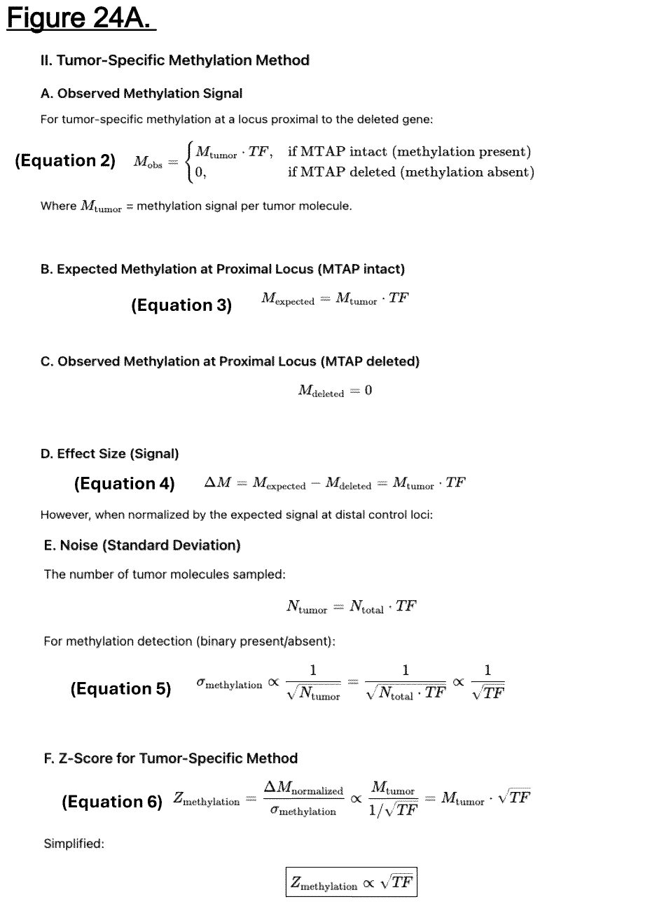

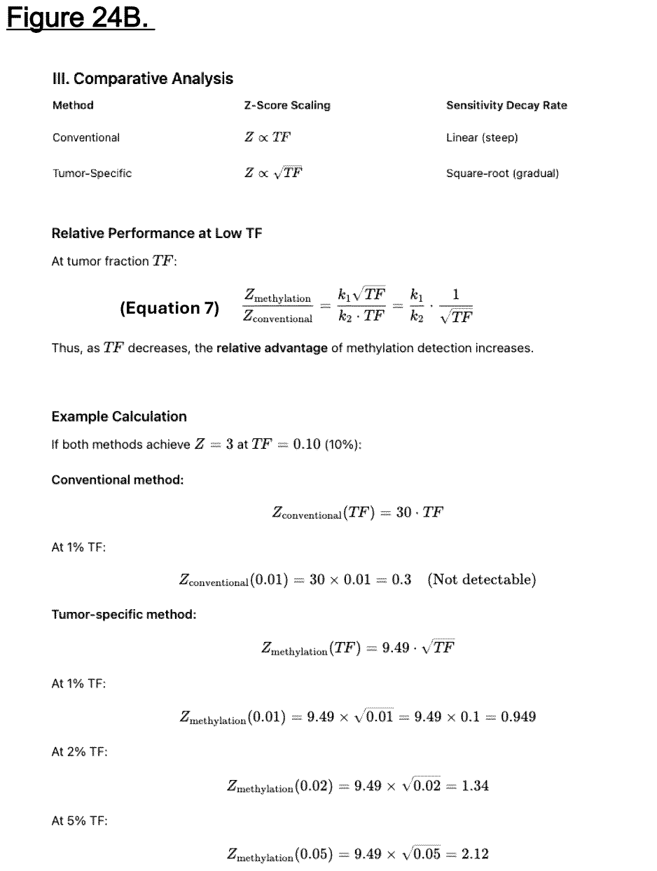

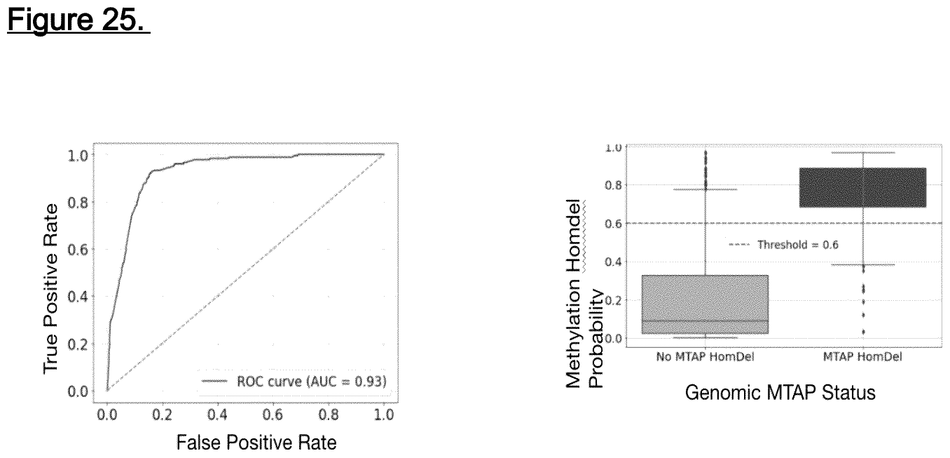

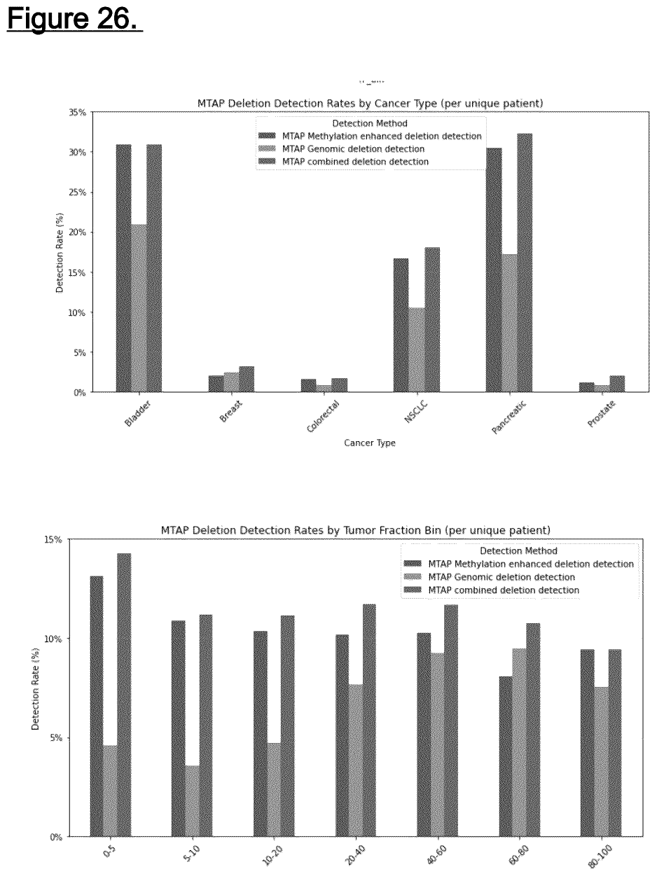

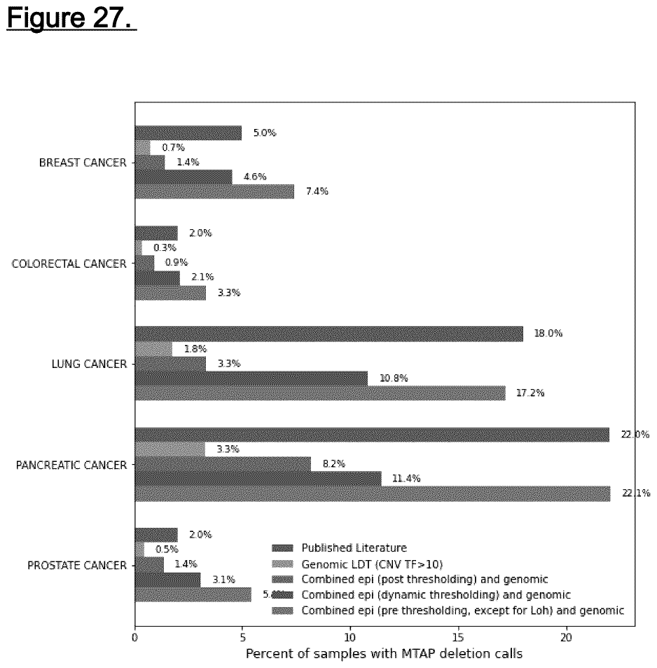

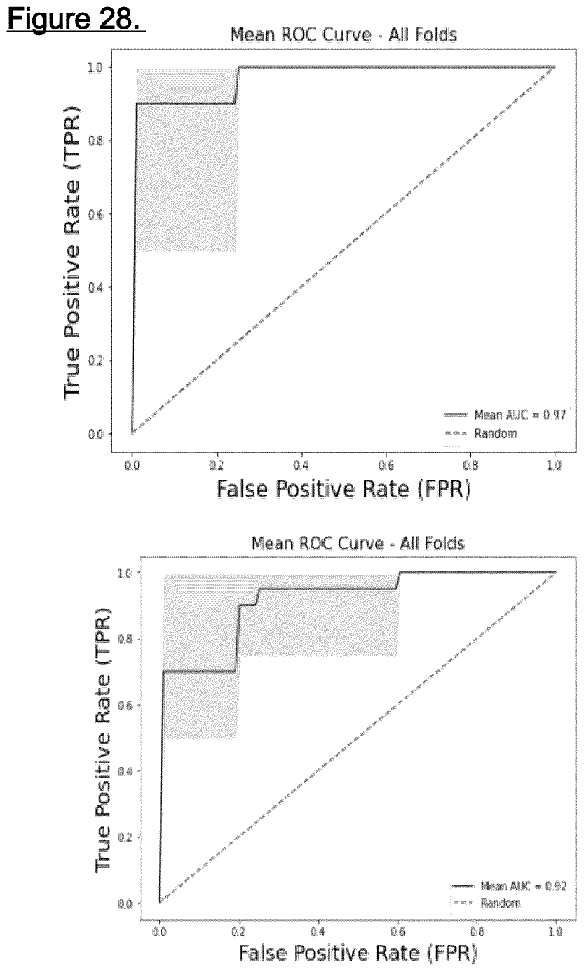

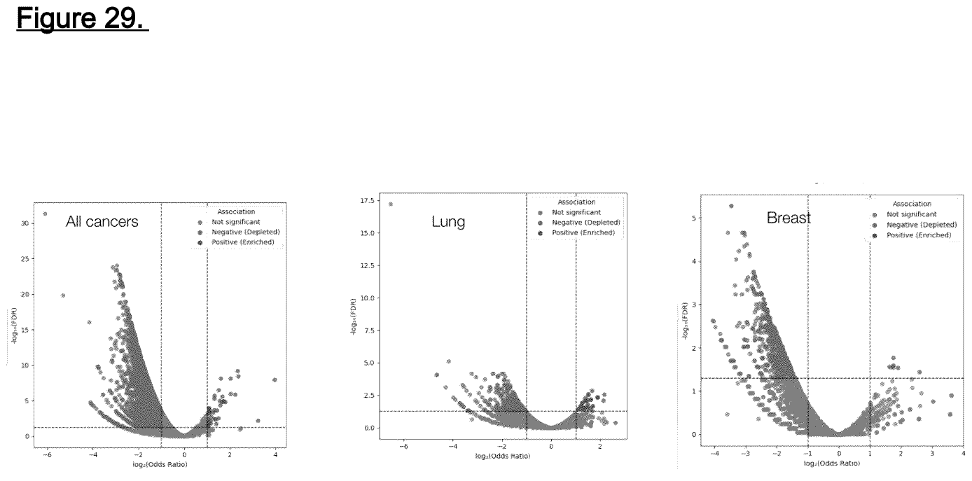

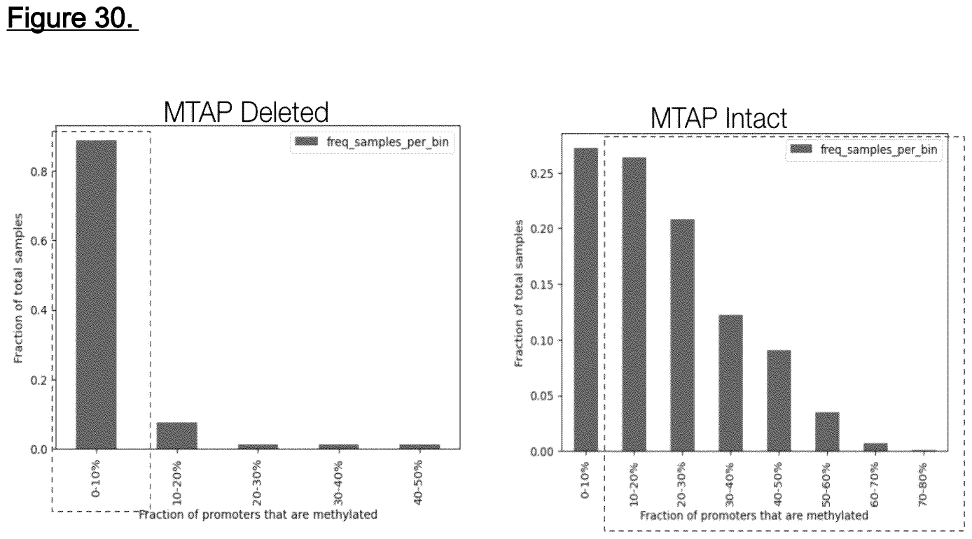

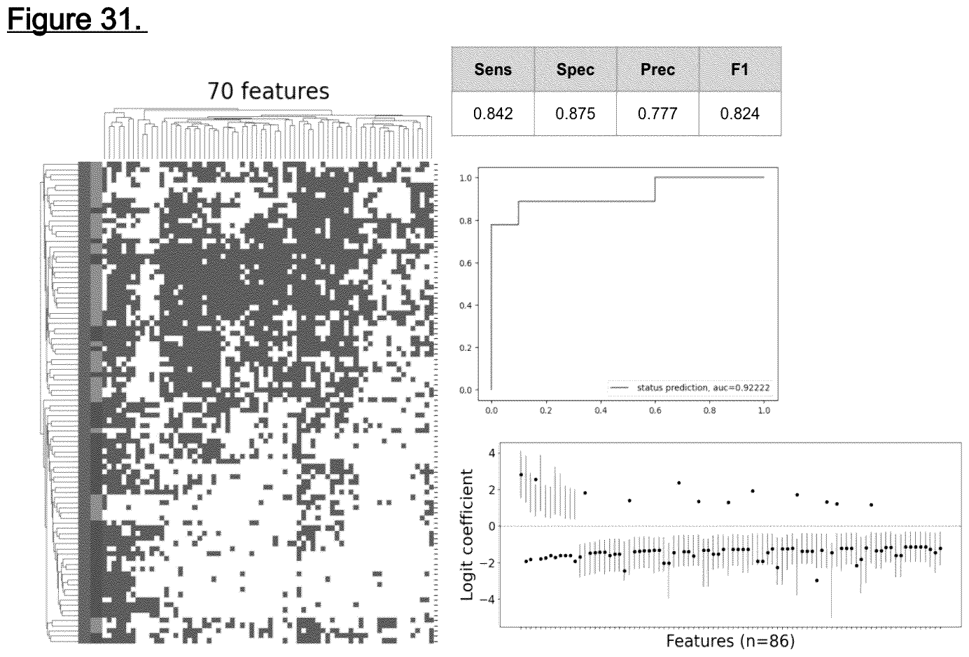

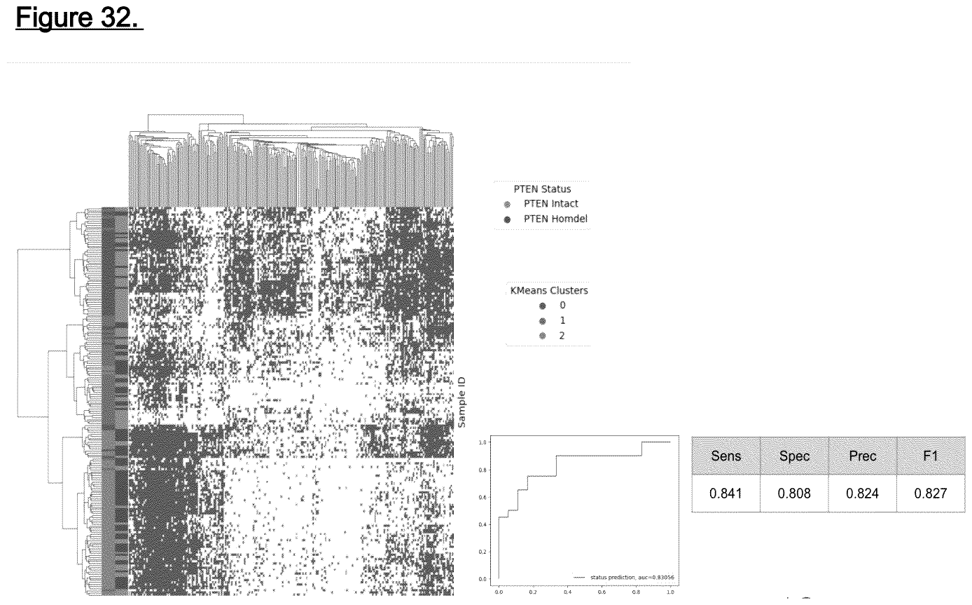

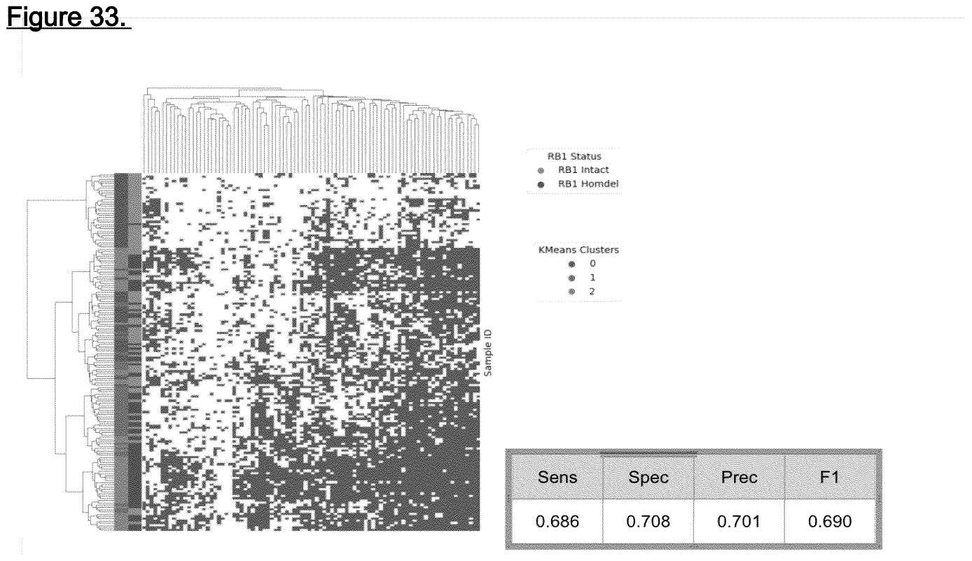

As shown in FIG. 9, the validation study demonstrates strong concordance between the methylation-enhanced method and tissue NGS as an orthogonal truth for MTAP HomDel status.

### Analytes

Analytes can include nucleic acid analytes, and non-nucleic acid analytes. The disclosure provides for detecting genetic variations in biological samples from a subject. Biological samples may include polynucleotides from cancer cells. Polynucleotides may be DNA (e.g., genomic DNA, cDNA), RNA (e.g., mRNA, small RNAs), or any combination thereof. Biological samples may include tumor tissue, e.g., from a biopsy. In some cases, biological samples may include blood or saliva. In particular cases, biological samples may comprise cell free DNA (“cfDNA”) or circulating tumor DNA (“ctDNA”). Cell free DNA can be present in, e.g., blood.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquity lati on variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. This further includes receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherents junction.

In general, the systems, apparatus, methods, and compositions can be used to analyze any number of analytes, further including both nucleic acid analytes and non-nucleic acid analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate. Methods for performing multiplexed assays to analyze two or more different analytes will be discussed in a subsequent section of this disclosure.

One or more nucleic acid analytes and/or non-nucleic acid analytes constitute a set of molecular interactions in a biological system under study (e.g., cells), which may be regarded as “interactome”—the molecular interactions that occur between molecules belonging to different biochemical families (proteins, nucleic acids, lipids, carbohydrates, etc.) and also within a given family. In various embodiments, an interactome is a protein-DNA interactome (network formed by transcription factors (and DNA or chromatin regulatory proteins) and their target genes. In other embodiments, interactome refers to protein-protein interaction network (PPI), or protein interaction network (PIN). The methods described herein allow for study and analysis of the interactome. Techniques such as proteogenomics (whole genome sequencing, whole exome sequencing and RNA-seq, and mass spectrometry as examples) can support study of the interactome.

### Multi-Omic Integration

In another embodiments, the present invention can rely on such tumor-specific methylation analytical method for integration of tumor-specific methylation predictions with conventional genomic copy number analysis. In various embodiment, The combined multi-modal approach improves both sensitivity and specificity over either method alone, and further allows for analysis for low analytes which otherwise would lack informative signal alone, but can provide informative signal when drawing upon an additional multi-modal detection, which can be regarded as a form of real-time orthogonal measure.

In one example, an integration is performed through a weighted ensemble:

P_final=w₁×P_methylation+w₂×P_coverage

where weights are determined by cross-validation to optimize performance. This includes, for example, a multi-omic classifier that demonstrates superior accuracy compared to single-modality approaches. Optionally, in various embodiments, one can combined additional signal through the union of high confidence MTAP HomDel from genomic or methylation for the purposes of final MTAP HomDel classification. In one example, integration enables detection of a homozygous deletion, including MTAP HomDel, at over 2.4-fold higher sensitivity than genomic-only methods, with particular advantages in low tumor fraction samples where conventional methods fail.

### Analysis

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic and other analyte data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

The present analyses are also useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The present methods can also be used for detecting genetic variations in conditions other than cancer. Immune cells, such as B cells, may undergo rapid clonal expansion upon the presence of certain diseases. Clonal expansions may be monitored using copy number variation detection and certain immune states may be monitored. In this example, copy number variation analysis may be performed over time to produce a profile of how a particular disease may be progressing. Copy number variation or even rare mutation detection may be used to determine how a population of pathogens changes during the course of infection. This may be particularly important during chronic infections, such as HIV/AIDS or Hepatitis infections, whereby viruses may change life cycle state and/or mutate into more virulent forms during the course of infection. The present methods may be used to determine or profile rejection activities of the host body, as immune cells attempt to destroy transplanted tissue to monitor the status of transplanted tissue as well as altering the course of treatment or prevention of rejection.

Further, the methods of the disclosure may be used to characterize the heterogeneity of an abnormal condition in a subject. Such methods can include, e.g., generating a genetic profile of extracellular polynucleotides derived from the subject, wherein the genetic profile includes a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation and mutation analyses alone or in combination.

The present methods can be used to diagnose, prognose, monitor or observe cancers. or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

### Determining Sensitivity at Low Tumor Fraction

An important advantage of the tumor-specific methylation approach is its superior performance at low tumor fractions compared to conventional coverage-based methods.

The observation that mutual exclusivity can rely on proximal loci to be informative, a mathematical calculation for this improvement is as follows:

In conventional methods, total sequencing coverage includes both tumor and normal DNA. For a homozygous deletion, the observed coverage is:

Conventional Coverage Method: C_obs=2(1−TF)+0(TF)=2−2TF (Equation 1)

where TF is tumor fraction. The coverage drop is proportional to TF, meaning the signal vanishes linearly as tumor fraction decreases.

In contrast, tumor-specific methylation methods isolate tumor DNA signals. The effect size (presence vs. absence of methylation) remains constant regardless of tumor fraction. The signal-to-noise ratio scales with √TF rather than TF, because only the noise increases with reduced molecule count while the effect size stays constant (2 copies→0 copies in tumor cells). This yields a more gradual decay of sensitivity as tumor fraction decreases, resulting in detection capability at much lower tumor fractions than conventional methods. In this aspect, described the tumor-specific method maintains a more linear relationship between detection power and tumor fraction, whereas conventional methods show a steep drop-off.

In various embodiments analytical approach applies to various instances in which there is a low amount of analyte, including rare analytes. For example, this includes but is not limited to low-shedding cfDNA samples, also to low ng input tissue biopsies, low tumor purity tissue samples where the absolute number of tumor DNA molecules is limited.

### Limit of Blank and Limit of Detection

Limit of blank (LoB) specificity was established using 120 cancer-free donor cfDNA samples (30 ng input). No false positives were observed among these cancer-free donors, confirming high specificity.

The 95% limit of detection (LoD) was determined via in silico dilutions of 50 high-confidence MTAP HomDel samples. Greater than 85% NPA was achieved down to at least 1% tumor fraction. The receiver operating characteristic curve demonstrates excellent discrimination between MTAP HomDel and MTAP-intact samples across a range of tumor fractions.

Mathematical modeling demonstrates that tumor-specific methylation methods achieve superior sensitivity at low tumor fractions compared to conventional coverage-based methods, with signal-to-noise ratio scaling as √TF rather than TF. Multi-omic integration of methylation and genomic features further enhances performance. The methods detect MTAP homozygous deletion (whole gene deletion or partial gene deletion) with AUC of 0.93, PPV of 100%, and NPA of 100% above limit of detection, achieving over 2.4-fold improvement in sensitivity compared to genomic-only methods.

### Determination of 5-methylcytosine Pattern of Nucleic Acids

Bisulfite-based sequencing and variants thereof provides a means of determining the methylation pattern of a nucleic acid. In some embodiments, determining the methylation pattern includes distinguishing 5-methylcytosine (5mC) from non-methylated cytosine. In some embodiments, determining methylation pattern includes distinguishing N6-methyladenine from non-methylated adenine. In some embodiments, determining the methylation pattern includes distinguishing 5-hydroxymethylcytosine (5hmC), 5-formylcytosine (5fC), and 5-carboxylcytosine (5caC) from non-methylated cytosine. Examples of bisulfite sequencing include, but are not limited to oxidative bisulfite sequencing (OX-BS-seq), Tet-assisted bisulfite sequencing (TAB-seq), and reduced bisulfite sequencing (redBS-seq).

Oxidative bisulfite sequencing (OX-BS-seq) is used to distinguish between 5mC and 5hmC, by first converting the 5hmC to 5fC, and then proceeding with bisulfite sequencing as previously described. Tet-assisted bisulfite sequencing (TAB-seq) can also be used to distinguish 5mc and 5hmC. In TAB-seq, 5hmC is protected by glucosylation. A Tet enzyme is then used to convert 5mC to 5caC before proceeding with bisulfite sequencing, as previously described. Reduced bisulfite sequencing is used to distinguish 5fC from modified cytosines.

Generally, in bisulfite sequencing, a nucleic acid sample is divided into two aliquots and one aliquot is treated with bisulfite. The bisulfite converts native cytosine and certain modified cytosine nucleotides (e.g. 5-formylcytosine or 5-carboxylcytosine) to uracil whereas other modified cytosines (e.g., 5-methylcytosine, 5-hydroxylmethylcystosine) are not converted.

Comparison of nucleic acid sequences of molecules from the two aliquots indicates which cytosines were and were not converted to uracils. Consequently, cytosines which were and were not modified can be determined. The initial splitting of the sample into two aliquots is disadvantageous for samples containing only small amounts of nucleic acids, and/or composed of heterogeneous cell/tissue origins such as bodily fluids containing cell-free DNA.

The present disclosure provides methods allowing bisulfite sequencing and variants thereof. These methods work by linking nucleic acids in a population to a capture moiety, i.e., a label that can be captured or immobilized. Capture moieties include, without limitation, biotin, avidin, streptavidin, a nucleic acid including a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase. Following linking of capture moieties to sample nucleic acids, the sample nucleic acids serve as templates for amplification. Following amplification, the original templates remain linked to the capture moieties, but amplicons are not linked to capture moieties.

The capture moiety can be linked to sample nucleic acids as a component of an adapter, which may also provide amplification and/or sequencing primer binding sites. In some methods, sample nucleic acids are linked to adapters at both ends, with both adapters bearing a capture moiety. Preferably any cytosine residues in the adapters are modified, such as by 5methylcytosine, to protect against the action of bisulfite. In some instances, the capture moieties are linked to the original templates by a cleavable linkage (e.g., photocleavable desthiobiotin-TEG or uracil residues cleavable with USER™ enzyme, Chem. Commun. (Camb). 2015 Feb. 21; 51(15): 3266-3269), in which case the capture moieties can, if desired, be removed.

The amplicons are denatured and contacted with an affinity reagent for the capture tag. Original templates bind to the affinity reagent whereas nucleic acid molecules resulting from amplification do not. Thus, the original templates can be separated from nucleic acid molecules resulting from amplification.

Following separation or partition, the respective populations of nucleic acids (i.e., original templates and amplification products) can be subjected to bisulfite treatment with the original template population receiving bisulfite treatment and the amplification products not. Alternatively, the amplification products can be subjected to bisulfite treatment and the original template population is not. Following such treatment, the respective populations can be amplified (which in the case of the original template population converts uracils to thymines). The populations can also be subjected to biotin probe hybridization for enrichment. The respective populations are then analyzed and sequences compared to determine which cytosines were 5-methylated (or 5-hydroxylmethylated) in the original. Detection of a T nucleotide in the template population (corresponding to an unmethylated cytosine converted to uracil) and a C nucleotide at the corresponding position of the amplified population indicates an unmodified C. The presence of C's at corresponding positions of the original template and amplified populations indicates a modified C in the original sample.

In some embodiments, a method uses sequential DNA-seq and bisulfite-seq (BIS-seq) NGS library preparation of molecular tagged DNA libraries. This process is performed by labeling of adapters (e.g., biotin), DNA-seq amplification of whole library, parent molecule recovery (e.g. streptavidin bead pull down), bisulfite conversion and BIS-seq. In some embodiments, the method identifies 5-methylcytosine with single-base resolution, through sequential NGS-preparative amplification of parent library molecules with and without bisulfite treatment. This can be achieved by modifying the 5-methyl-ated NGS-adapters (directional adapters; Y-shaped/forked with 5-methylcytosine replacing) used in BIS-seq with a label (e.g., biotin) on one of the two adapter strands. Sample DNA molecules are adapter ligated, and amplified (e.g., by PCR). As only the parent molecules will have a labeled adapter end, they can be selectively recovered from their amplified progeny by label-specific capture methods (e.g., streptavidin-magnetic beads). As the parent molecules retain 5-methylation marks, bisulfite conversion on the captured library will yield single-base resolution 5-methylation status upon BIS-seq, retaining molecular information to corresponding DNA-seq. In some embodiments, the bisulfite treated library can be combined with a non-treated library prior to enrichment/NGS by addition of a sample tag DNA sequence in standard multiplexed NGS workflow. As with BIS-seq workflows, bioinformatics analysis can be carried out for genomic alignment and 5-methylated base identification. In sum, this method provides the ability to selectively recover the parent, ligated molecules, carrying 5-methylcytosine marks, after library amplification, thereby allowing for parallel processing for bisulfite converted DNA. This overcomes the destructive nature of bisulfite treatment on the quality/sensitivity of the DNA-seq information extracted from a workflow. With this method, the recovered ligated, parent DNA molecules (via labeled adapters) allow amplification of the complete DNA library and parallel application of treatments that elicit epigenetic DNA modifications. The present disclosure discusses the use of BIS-seq methods to identify cytosine5-methylation (5-methylcytosine), but this is not limiting. Variants of BIS-seq have been developed to identify hydroxymethylated cytosines (5hmC; OX-BS-seq, TAB-seq), formylcytosine (5fC; redBS-seq) and carboxylcytosines. These methodologies can be implemented with the sequential/parallel library preparation described herein.

### Alternative Methods of Modified Nucleic Acid Analysis

The disclosure provides alternative methods for analyzing modified nucleic acids (e.g., methylated, linked to histones and other modifications discussed above). In some such methods, a population of nucleic acids bearing the modification to different extents (e.g., 0, 1, 2, 3, 4, 5 or more methyl groups per nucleic acid molecule) is contacted with adapters before fractionation of the population depending on the extent of the modification. Adapters attach to either one end or both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. Following attachment of adapters, the nucleic acids are amplified from primers binding to the primer binding sites within the adapters. Adapters, whether bearing the same or different tags, can include the same or different primer binding sites, but preferably adapters include the same primer binding site. Following amplification, the nucleic acids are contacted with an agent that preferably binds to nucleic acids bearing the modification (such as the previously described such agents). The nucleic acids are separated into at least two partitions differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. Following separation, the different partitions can then be subject to further processing steps, which typically include further amplification, and sequence analysis, in parallel but separately. Sequence data from the different partitions can then be compared.

Nucleic acids can be linked at both ends to Y-shaped adapters including primer binding sites and tags. The molecules are amplified. The amplified molecules are then fractionated by contact with an antibody preferentially binding to 5-methylcytosine to produce two partitions. One partition includes original molecules lacking methylation and amplification copies having lost methylation. The other partition includes original DNA molecules with methylation. The two partitions are then processed and sequenced separately with further amplification of the methylated partition. The sequence data of the two partitions can then be compared. In this example, tags are not used to distinguish between methylated and unmethylated DNA but rather to distinguish between different molecules within these partitions so that one can determine whether reads with the same start and stop points are based on the same or different molecules.

The disclosure provides further methods for analyzing a population of nucleic acid in which at least some of the nucleic acids include one or more modified cytosine residues, such as 5-methylcytosine and any of the other modifications described previously. In these methods, the population of nucleic acids is contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are treated with bisulfite. This treatment converts unmodified cytosines to uracils. The bisulfite treated nucleic acids are then subjected to amplification primed by primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

### Partitioning the Sample Into a Plurality of Subsamples; Aspects of Samples; Analysis of Epigenetic Characteristics

In certain embodiments described herein, a population of different forms of nucleic acids (e.g., hypermethylated and hypomethylated DNA in a sample, such as a captured set of cfDNA as described herein) can be physically partitioned based on one or more characteristics of the nucleic acids prior to further analysis, e.g., differentially modifying or isolating a nucleobase, tagging, and/or sequencing. This approach can be used to determine, for example, whether certain sequences are hypermethylated or hypomethylated. In some embodiments, hypermethylation variable epigenetic target regions are analyzed to determine whether they show hypermethylation characteristic of tumor cells and/or hypomethylation variable epigenetic target regions are analyzed to determine whether they show hypomethylation characteristic of tumor cells. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some instances, a heterogeneous nucleic acid sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein) and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially labelled, and the partitions are pooled together prior to sequencing. In other instances, the different forms are separately sequenced. In some embodiments, a population of different nucleic acids is partitioned into two or more different partitions. Each partition is representative of a different nucleic acid form, and a first partition (also referred to as a subsample) includes DNA with a cytosine modification in a greater proportion than a second subsample. Each partition is distinctly tagged. The first subsample is subjected to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. The tagged nucleic acids are pooled together prior to sequencing. Sequence reads are obtained and analyzed, including to distinguish the first nucleobase from the second nucleobase in the DNA of the first subsample, in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Samples can include nucleic acids varying in modifications including post-replication modifications to nucleotides and binding, usually noncovalently, to one or more proteins.

In an embodiment, the population of nucleic acids is one obtained from a serum, plasma or blood sample from a subject suspected of having neoplasia, a tumor, or cancer or previously diagnosed with neoplasia, a tumor, or cancer. The population of nucleic acids includes nucleic acids having varying levels of methylation. Methylation can occur from any one or more post-replication or transcriptional modifications. Post-replication modifications include modifications of the nucleotide cytosine, particularly at the 5-position of the nucleobase, e.g., 5-methylcytosine, 5-hydroxymethylcytosine, 5-formylcytosine and 5-carboxylcytosine. The affinity agents can be antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target.

Examples of capture moieties contemplated herein include methyl binding domain (MBDs) and methyl binding proteins (MBPs) as described herein, including proteins such as MeCP2 and antibodies preferentially binding to 5-methylcytosine. Likewise, partitioning of different forms of nucleic acids can be performed using histone binding proteins which can separate nucleic acids bound to histones from free or unbound nucleic acids. Examples of histone binding proteins that can be used in the methods disclosed herein include RBBP4, RbAp48 and SANT domain peptides. Although for some affinity agents and modifications, binding to the agent may occur in an essentially all or none manner depending on whether a nucleic acid bears a modification, the separation may be one of degree. In such instances, nucleic acids overrepresented in a modification bind to the agent at a greater extent that nucleic acids underrepresented in the modification. Alternatively, nucleic acids having modifications may bind in an all or nothing manner. But then, various levels of modifications may be sequentially eluted from the binding agent.

For example, in some embodiments, partitioning can be binary or based on degree/level of modifications. For example, all methylated fragments can be partitioned from unmethylated fragments using methyl-binding domain proteins (e.g., MethylMiner Methylated DNA Enrichment Kit (ThermoFisher Scientific)). Subsequently, additional partitioning may involve eluting fragments having different levels of methylation by adjusting the salt concentration in a solution with the methyl-binding domain and bound fragments. As salt concentration increases, fragments having greater methylation levels are eluted. In some instances, the final partitions are representative of nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MethylMiner Methylated DNA Enrichment Kit (ThermoFisher Scientific) various levels of methylation can be partitioned using sequential elutions. For example, a hypomethylated partition (e.g., no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, at least 300 mM, at least 400 mM, at least 500 mM, at least 600 mM, at least 700 mM, at least 800 mM, at least 900 mM, at least 1000 mM, or at least 2000 mM. After such methylated nucleic acids are eluted, magnetic separation is once again used to separate higher levels of methylated nucleic acids from those with lower level of methylation. The elution and magnetic separation steps can repeat themselves to create various partitions such as a hypomethylated partition (representative of no methylation), a methylated partition (representative of low level of methylation), and a hyper methylated partition (representative of high level of methylation).

In some methods, nucleic acids bound to an agent used for affinity separation are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent). The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition. For further details regarding portioning nucleic acid samples based on characteristics such as methylation, see WO2018/119452, which is incorporated herein by reference. In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, partitioning of the nucleic acids is performed by contacting the nucleic acids with a methylation binding domain (“MBD”) of a methylation binding protein (“MBP”). MBD binds to 5-methylcytosine (5mC). MBD is coupled to paramagnetic beads, such as Dynabeads® M-280 Streptavidin via a biotin linker. Partitioning into fractions with different extents of methylation can be performed by eluting fractions by increasing the NaCl concentration.

An exemplary method for molecular tag identification of MBD-bead partitioned libraries through NGS is as follows:

Physical partitioning of an extracted DNA sample (e.g., extracted blood plasma DNA from a human sample) using a methyl-binding domain protein-bead purification kit, saving all elutions from process for downstream processing.

Parallel application of differential molecular tags and NGS-enabling adapter sequences to each partition. For example, the hypermethylated, residual methylation (‘wash’), and hypomethylated partitions are ligated with NGS-adapters with molecular tags.

Re-combining all molecular tagged partitions, and subsequent amplification using adapter-specific DNA primer sequences.

Enrichment/hybridization of re-combined and amplified total library, targeting genomic regions of interest (e.g., cancer-specific genetic variants and differentially methylated regions).

Re-amplification of the enriched total DNA library, appending a sample tag. Different samples are pooled and assayed in multiplex on an NGS instrument.

Bioinformatics analysis of NGS data, with the molecular tags being used to identify unique molecules, as well deconvolution of the sample into molecules that were differentially MBD-partitioned. This analysis can yield information on relative 5-methylcytosine for genomic regions, concurrent with standard genetic sequencing/variant detection.

Examples of MBPs contemplated herein include, but are not limited to:
(a) MeCP2 is a protein preferentially binding to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated nucleotide bases.

In general, elution is a function of number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nM to about 2500 mM NaCl. In one embodiment, the process results in three (3) partitions.

Molecules are contacted with a solution at a first salt concentration and including a molecule including a methyl binding domain, which molecule can be attached to a capture moiety, such as streptavidin. At the first salt concentration a population of molecules will bind to the MBD and a population will remain unbound. The unbound population can be separated as a “hypomethylated” population. For example, a first partition representative of the hypomethylated form of DNA is that which remains unbound at a low salt concentration, e.g., 100 mM or 160 mM. A second partition representative of intermediate methylated DNA is eluted using an intermediate salt concentration, e.g., between 100 mM and 2000 mM concentration. This is also separated from the sample. A third partition representative of hypermethylated form of DNA is eluted using a high salt concentration, e.g., at least about 2000 mM.

The disclosure provides further methods for analyzing a population of nucleic acids in which at least some of the nucleic acids include one or more modified cytosine residues, such as 5-methylcytosine and any of the other modifications described previously. In these methods, after partitioning, the subsamples of nucleic acids are contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. The nucleic acids subjected to the procedure are then amplified with primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Such an analysis can be performed using the following exemplary procedure. After partitioning, methylated DNA is linked to Y-shaped adapters at both ends including primer binding sites and tags. The cytosines in the adapters are modified at the 5 position (e.g., 5-methylated). The modification of the adapters serves to protect the primer binding sites in a subsequent conversion step (e.g., bisulfite treatment, TAP conversion, or any other conversion that does not affect the modified cytosine but affects unmodified cytosine). After attachment of adapters, the DNA molecules are amplified. The amplification product is split into two aliquots for sequencing with and without conversion. The aliquot not subjected to conversion can be subjected to sequence analysis with or without further processing. The other aliquot is subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. Only primer binding sites protected by modification of cytosines can support amplification when contacted with primers specific for original primer binding sites. Thus, only original molecules and not copies from the first amplification are subjected to further amplification. The further amplified molecules are then subjected to sequence analysis. Sequences can then be compared from the two aliquots. As in the separation scheme discussed above, nucleic acid tags in adapters are not used to distinguish between methylated and unmethylated DNA but to distinguish nucleic acid molecules within the same partition.

### Subjecting the First Subsample to a Procedure that Affects a First Nucleobase in the DNA Differently From a Second Nucleobase in the DNA of the First Subsample

Methods disclosed herein comprise a step of subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases includes mC and the other includes hmC.

In some embodiments, the procedure that affects a first nucleobase in the DNA differently from a second nucleobase in the DNA of the first subsample includes bisulfite conversion. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase includes one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion on a first subsample as described herein thus facilitates identifying positions containing mC or hmC using the sequence reads obtained from the first subsample. For an exemplary description of bisulfite conversion, see, e.g., Moss et al., Nat Commun. 2018; 9: 5068.

In some embodiments, the procedure that affects a first nucleobase in the DNA differently from a second nucleobase in the DNA of the first subsample includes oxidative bisulfite (Ox-BS) conversion. In some embodiments, the procedure that affects a first nucleobase in the DNA differently from a second nucleobase in the DNA of the first subsample includes Tet-assisted bisulfite (TAB) conversion. In some embodiments, the procedure that affects a first nucleobase in the DNA differently from a second nucleobase in the DNA of the first subsample includes Tet-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes chemical-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes APOBEC-coupled epigenetic (ACE) conversion.

In some embodiments, procedure that affects a first nucleobase in the DNA differently from a second nucleobase in the DNA of the first subsample includes enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-BGT can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA of the first subsample includes separating DNA originally including the first nucleobase from DNA not originally including the first nucleobase.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N6-methyladenine (mA), N6-hydroxymethyladenine (hmA), or N6-formyladenine (fA).

Techniques including methylated DNA immunoprecipitation (MeDIP) can be used to separate DNA containing modified bases such as mA from other DNA. See, e.g., Kumar et al., Frontiers Genet. 2018; 9:640; Greer et al., Cell 2015; 161:868-878. An antibody specific for mA is described in Sun et al., Bioessays 2015; 37:1155-62. Antibodies for various modified nucleobases, such as forms of thymine/uracil including halogenated forms such as 5-bromouracil, are commercially available. Various modified bases can also be detected based on alterations in their base-pairing specificity. For example, hypoxanthine is a modified form of adenine that can result from deamination and is read in sequencing as a G. See, e.g., U.S. Pat. No. 8,486,630; Brown, Genomes, 2nd Ed., John Wiley & Sons, Inc., New York, N.Y., 2002, chapter 14, “Mutation, Repair, and Recombination.”

### Enriching/Capturing Step, Amplification, Adaptors, Barcodes

In some embodiments, methods disclosed herein comprise a step of capturing one or more sets of target regions of DNA, such as cfDNA. Capture may be performed using any suitable approach known in the art. In some embodiments, capturing includes contacting the DNA to be captured with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. Where the first subsample undergoes a separation step (e.g., separating DNA originally including the first nucleobase (e.g., hmC) from DNA not originally including the first nucleobase, such as hmC-seal), capturing may be performed on any, any two, or all of the DNA originally including the first nucleobase (e.g., hmC), the DNA not originally including the first nucleobase, and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein includes capturing cfDNA obtained from a test subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules, and the cfDNA molecules corresponding to the sequence-variable target region set are captured at a greater capture yield in the captured set of cfDNA molecules than cfDNA molecules corresponding to the epigenetic target region set. For additional discussion of capturing steps, capture yields, and related aspects, see WO2020/160414, which is incorporated herein by reference for all purposes.

In some embodiments, a method described herein includes contacting cfDNA obtained from a test subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In various embodiments, the methods further comprise sequencing the captured cfDNA, e.g., to different degrees of sequencing depth for the epigenetic and sequence-variable target region sets, consistent with the discussion herein. In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some such embodiments, the capturing step is performed with the probes for the sequence-variable target region set and the probes for the epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions including captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, the DNA is amplified. In some embodiments, amplification is performed before the capturing step. In some embodiments, amplification is performed after the capturing step.

In some embodiments, adapters are included in the DNA. This may be done concurrently with an amplification procedure, e.g., by providing the adapters in a 5′ portion of a primer, e.g., as described above. Alternatively, adapters can be added by other approaches, such as ligation.

In some embodiments, tags, which may be or include barcodes, are included in the DNA. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described above. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed in the “General Features of the Methods” section below, which can be combined to the extent practicable with any of the foregoing embodiments and the embodiments set forth in the introduction and summary section.

### Therapies and Related Administration

In certain embodiments, the methods disclosed herein relate to identifying and administering customized therapies to patients given the status of a nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the test subject and/or patients who are receiving, or who have received, the same therapy as the test subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraarticular, which administration may include tablets, capsules, granules, aqueous suspensions, gels, sprays, suppositories, salves, ointments, or the like.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it should be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

### Clinical Implementation

The MTAP HomDel detection method using tumor-specific methylation is now implemented as a commercial product. Assessment of consecutive samples tested in routine clinical care demonstrates that the multi-omic MTAP HomDel detection achieves detection rates consistent with tissue-based testing and literature reports, with over 2.4-fold improvement compared to genomic-only methods.

The methodology enables identification of patients eligible for PRMT5 and MAT2A inhibitor therapies, which demonstrate synthetic lethality in MTAP-deleted tumors. This has significant clinical impact for patient selection in clinical trials and for therapeutic decision-making.

### Genetic Analysis

Genetic analysis includes detection of nucleotide sequence variants and copy number variations. Genetic variants can be determined by sequencing. The sequencing method can be massively parallel sequencing, that is, simultaneously (or in rapid succession) sequencing any of at least 100,000, 1 million, 10 million, 100 million, or 1 billion polynucleotide molecules. Sequencing methods may include, but are not limited to: high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next-generation sequencing, Single Molecule Sequencing by Synthesis (SMSS) (Helicos), massively-parallel sequencing, Clonal Single Molecule Array (Solexa), shotgun sequencing, Maxam-Gilbert or Sanger sequencing, primer walking, sequencing using PacBio, SOLID, Ion Torrent, or Nanopore platforms and any other sequencing methods known in the art.

Sequencing can be made more efficient by performing sequence capture, that is, the enrichment of a sample for target sequences of interest, e.g., sequences including the KRAS and/or EGFR genes or portions of them containing sequence variant biomarkers. Sequence capture can be performed using immobilized probes that hybridize to the targets of interest.

Cell free DNA can include small amounts of tumor DNA mixed with germline DNA. Sequencing methods that increase sensitivity and specificity of detecting tumor DNA, and, in particular, genetic sequence variants and copy number variation, can be useful in the methods of this invention. Such methods are described in, for example, in WO 2014/039556. These methods not only can detect molecules with a sensitivity of up to or greater than 0.1%, but also can distinguish these signals from noise typical in current sequencing methods. Increases in sensitivity and specificity from blood-based samples of cfDNA can be achieved using various methods. One method includes high efficiency tagging of DNA molecules in the sample, e.g., tagging at least any of 50%, 75% or 90% of the polynucleotides in a sample. This increases the likelihood that a low-abundance target molecule in a sample will be tagged and subsequently sequenced, and significantly increases sensitivity of detection of target molecules.

Another method involves molecular tracking, which identifies sequence reads that have been redundantly generated from an original parent molecule, and assigns the most likely identity of a base at each locus or position in the parent molecule. This significantly increases specificity of detection by reducing noise generated by amplification and sequencing errors, which reduces frequency of false positives.

Methods of the present disclosure can be used to detect genetic variation in non-uniquely tagged initial starting genetic material (e.g., rare DNA) at a concentration that is less than 5%, 1%, 0.5%, 0.1%, 0.05%, or 0.01%, at a specificity of at least 99%, 99.9%, 99.99%, 99.999%, 99.9999%, or 99.99999%. Sequence reads of tagged polynucleotides can be subsequently tracked to generate consensus sequences for polynucleotides with an error rate of no more than 2%, 1%, 0.1%, or 0.01%.

Copy number variation determination can involve determining a quantitative measure of polynucleotides in a sample mapping to a genetic locus, such as the EGFR gene or KRAS gene. The quantitative measure can be a number. Once the total number of polynucleotides mapping to a locus is determined, this number can be used in standard methods of determining Copy Number Variation at the locus. A quantitative measure can be normalized against a standard. In one method, a quantitative measure at a test locus can be standardized against a quantitative measure of polynucleotides mapping to a control locus in the genome, such as gene of known copy number. In another method, the quantitative measure can be compared against the amount of nucleic acid in the original sample. For example, the quantitative measure can be compared against an expected measure for diploidy. In another method, the quantitative measure can be normalized against a measure from a control sample, and normalized measures at different loci can be compared. In another method, quantifying involves quantifying parent or original molecules in a sample mapping to a locus, rather than number of sequence reads. A copy number variation may be an amplification or a deletion or truncation of a gene. An amplification may be 3, 4, 5, 6, 7, 8, 9, 10, or 10 or more copies of a gene. A deletion or truncation may be 0 or 1 copies of a gene.

An example of a method for detecting copy number variation may include an array. The array may comprise a plurality of capture probes. The capture probes can be oligonucleotides that are bound to the surface of the array. The capture probes may hybridize to at least one of the genes as set forth in Table 1. The capture probes may bind to at least 1, 2, 3, 4, 5, 6, 7, 8, 9, 10, 11, or 12 genes as set forth in Table 1. DNA derived from the subject may be labeled (e.g., with a fluorophore) prior to hybridization for detection.

### Detection of Single-Copy Number Loss

It is readily appreciated that the above techniques, including tumor specific methylation detection, is generally applicable to any genetic or function variant of interest, including for example, homodel, indel, fusion, repeats, single point mutation, frameshifts, or other loss or alteration in function. In various embodiments, this can include changes related to homologous recombination deficiency (HRD), allelic imbalance (Al), microsatellite instability (MSI) For example, in addition to homozygous deletion detection, the invention provides methods for detecting single-copy number loss (loss of heterozygosity, LOH). Single-copy loss results in reduction but not complete absence of tumor-specific methylation at proximal loci.

Further, the discrimination provided by the above analytical detection techniques allows a form of copy number variation (CNV) at a much higher degree of sensitivity. For example, the Inventors established separate models are trained for LOH detection using samples with confirmed single-copy loss. The LOH model utilizes 68 features after feature selection and cross-validation. This enables finer resolution of copy number status than conventional CNV methods, distinguishing between intact (2 copies), LOH (1 copy), and HomDel (0 copies).

### Principles of Tumor-Specific Methylation-Based Deletion Detection

The present invention exploits the mutual exclusivity between tumor-specific methylation and homozygous gene deletion. When both copies of a gene are deleted in tumor cells, the DNA sequence at that locus is absent, and therefore tumor-specific methylation cannot occur at that location. This principle enables detection of homozygous deletions through the absence of expected tumor-specific methylation signals at loci proximal to the deleted gene. For example, in samples with intact gene copies, tumor-specific methylation is observed at characteristic loci near the gene. In samples with homozygous deletion, this methylation signal disappears completely, while methylation at distal control loci remains present, providing an internal reference. It is appreciated by one of ordinary skill that mutual exclusivity may further operate in a probabilistic manner, such as a likelihood ratio, or other statistical function.

### Genome-Wide Methylation Changes in MTAP-Deleted Tumors

The invention further exploits the observation that genetic variants including for example, homodel, indel, fusion, repeats, single point mutation, frameshifts, or other loss or alteration in function are likely to manifest themselves in methylome alterations. In this aspect, methylation profiling via epigenome detection can be utilized to detect the underlying genetic variant at the genome level (e.g., epigenotyping). Importantly, this further includes gain of function, loss of function, or other functional alterations that may manifest similarly to a genetic variant, but not due to a genomic mutation, thereby identifying subjects that could benefit from similar therapies as arising from similar function deficiencies.

For example, MTAP deletion leads to genome-wide changes in DNA methylation patterns. In MTAP-deleted tumors, many loci exhibit reduced tumor-specific hypermethylation compared to MTAP-intact tumors. This phenomenon is related to the biological role of MTAP in the methionine salvage pathway. MTAP loss leads to partial disruption of PRMT5 activity, a methyltransferase that adds methyl groups to histones at specific locations. Modified chromatin by PRMT5 leads to gene silencing via DNA methyltransferase DNMT3A. MTAP loss is therefore associated with reduced DNA methylation of many promoters in at least some cancer types. In this aspect, any number of epigenetic regulators are likely to have particular pronounced effects, and are good candidates for epigenotyping identification.

The shift of these loci to lower methylation levels relative to other methylated loci provides quantitative scores that are incorporated as additional features for predicting copy number loss, further enhancing model performance.

Models for predicting genomic mutations (e.g., MTAP, PTEN, and RB1 HomDel) can utilized a data source such as TCGA 450K array data cross-referenced with an additional assay data source for training. These models show promising performance in predicting HomDel from liquid tissue biopsy and demonstrate applicability to cfDNA data using tumor-specific methylation features.

In other embodiments, described herein are methods for detecting driver mutations using methylation predicting the presence of genetic variants, genomic mutations, such as clinically relevant SNVs and indels using tumor-specific methylation patterns. In various embodiments, changes in tumor-specific methylation at specific loci are indicative of the presence of driver mutations.

For example, using the aforementioned methods, including epigenotyping, EGFR driver mutation detection were identified using methylation.

Models were developed to predict the presence of EGFR driver mutations from tissue biopsies using TCGA 450K array data as input features. The models predict presence of: EGFR Exon 19 in-frame deletions, L858R variants, G719 variants, L861Q variants, among others. These mutations are known to be clinically significant and impact tumor cell biology.

### EXAMPLES

### Example 1—Discovery and Validation of Tumor-Specific Methylation Biomarkers

The invention was developed using comprehensive genomic and epigenomic profiling across multiple cohorts:
1. MRD Discovery Cohort: 14,895 minimal residual disease (MRD) plasma samples from patients after curative-intent treatment for early-stage tumors were analyzed to identify tumor-specific methylation at the 9p21 locus and other genomically relevant regions.
2. Late-Stage Discovery Cohort: 23,143 plasma samples from late-stage tumors were analyzed to identify which candidate biomarkers were informative for gene deletion status.
3. Independent Validation Cohort: Approximately 14,000 plasma samples from late-stage tumors were used to confirm the association of candidate biomarkers to deletion status.

All samples were profiled using broad coverage for tumor-specific methylation as well as comprehensive genomic analysis.

### Example 2—MTAP Homozygous Deletion Detection From CFDNA

Plasma samples were collected from patients with advanced cancer and processed to extract cell-free DNA. Samples were analyzed using a platform for detecting genomic and epigenomic data.

Tumor-specific methylation was assessed at 46 promoter regions, including loci proximal to MTAP, CDKN2A, CDKN2A-DT, CDKN2B-AS1, DMRTA1, MIR31HG, HACD4 and distal control regions. A logistic regression model with elastic net regularization was applied to generate a probability score for MTAP HomDel.

In a validation cohort of 87 matched tissue-liquid samples, the methylation-enhanced method achieved 100% PPV and 100% NPA above the limit of detection. Samples with tumor fraction as low as 2.4% that were negative by conventional genomic methods were correctly identified as MTAP HomDel by the methylation-based approach.

### Example 3—MTAP Homozygous Deletion Biomarkers

Analysis of methylation frequency across 15,272 promoters in a cohort with tumor fraction between 10-20% identified 1,494 genes more frequently methylated in MTAP-intact samples compared to 256 genes more frequently methylated in MTAP-deleted samples (p<0.05, Fisher's exact test).

A subset of 127 promoters with odds ratio ≤0.2 and minimum frequency ≥10% in MTAP-intact samples was identified. In this promoter set, 89% of MTAP HomDel samples have 10% or less of these promoters methylated, while 73% of MTAP-intact samples have more than 10% methylated.

Six genes within 1 MB of the MTAP locus were identified as demonstrating cancer-specific hypermethylation: MTAP, CDKN2A, CDKN2A-DT, CDKN2B-AS1, DMRTA1, MIR31HG, and HACD4. Hypermethylation of regions next to all six candidate biomarkers was mutually exclusive with MTAP homozygous deletion relative to distal methylation sites.

As shown in FIG. 2, the conventional genomic method is dominated by non-tumor background noise at low tumor fractions, whereas the tumor-specific methylation method isolates tumor signals that dominate despite low tumor fraction.

### Example 4—Feature Selection

Input features for the predictive models include both binary and quantitative measures of tumor-specific methylation. Binary features indicate presence or absence of methylation at specific loci, while quantitative features provide normalized methylation levels.

For MTAP HomDel detection, univariate feature selection with logistic regression was performed, identifying features with p-value <0.01. The initial MTAP model utilized 46 cancer-specific methylated region features distributed across the human genome. An updated model with enhanced performance utilized 8 methylation regions as features after focusing on tumor-specific methylation coverage adjacent to the MTAP gene and additional training data and code optimization.

### Example 5—Model Architecture

Logistic regression models with elastic net regularization were trained to predict gene deletion status. The models were optimized through 5-fold cross-validation, with features having zero coefficients across all folds removed from the final model.

Training datasets were carefully balanced for tumor fraction and cancer type to avoid bias. For the MTAP HomDel model, training included 34 MTAP-loss samples and 44 MTAP-intact samples, achieving cross-validation metrics of: Sensitivity 0.89±0.14, Specificity 0.86±0.08, Precision 0.84±0.09, and F1 score 0.854±0.09.

The updated MTAP model with expanded training data (142 MTAP-loss and 117 MTAP-intact samples) achieved: Sensitivity 0.84±0.103, Specificity 0.86±0.048, Precision 0.83±0.047, and F1 score 0.832±0.054.

### Example 6—PTEN Homozygous Deletion Detection

A cohort of 322 samples including prostate (186), lung (70), and breast (66) cancers was analyzed for PTEN HomDel status. Tumor-specific methylation was assessed at 16 promoter regions identified through univariate feature selection.

A PTEN HomDel model was developed using 16 promoter methylation features, achieving performance metrics of: Sensitivity 0.841, Specificity 0.808, Precision 0.824, and F1 score 0.827 across a combined cohort of 322 samples including prostate (186), lung (70), and breast (66) cancers.

The PTEN HomDel model achieved sensitivity of 0.841, specificity of 0.808, precision of 0.824, and F1 score of 0.827. Performance was superior in prostate cancer alone (sensitivity 0.841, specificity 0.808) compared to the combined multi-cancer cohort, suggesting some cancer-type specificity in methylation patterns.

The methodology was extended to other clinically relevant genes:

### Example 7—Multi-Omic Integration for Enhanced Detection

Samples were analyzed using both conventional genomic copy number calling and tumor-specific methylation-based prediction and the union of high confidence MTAP HomDel by either genomic or methylation methods in order to make a final MTAP HomDel call. The two modalities were also integrated through a weighted ensemble approach in prototype.

The multi-omic classifier demonstrated 2.4-fold improvement in MTAP HomDel detection sensitivity compared to genomic-only methods. In low tumor fraction samples (TF<5%), the improvement was even more pronounced, with the multi-omic approach detecting deletions missed entirely by conventional methods.

### Example 8—Tissue Application

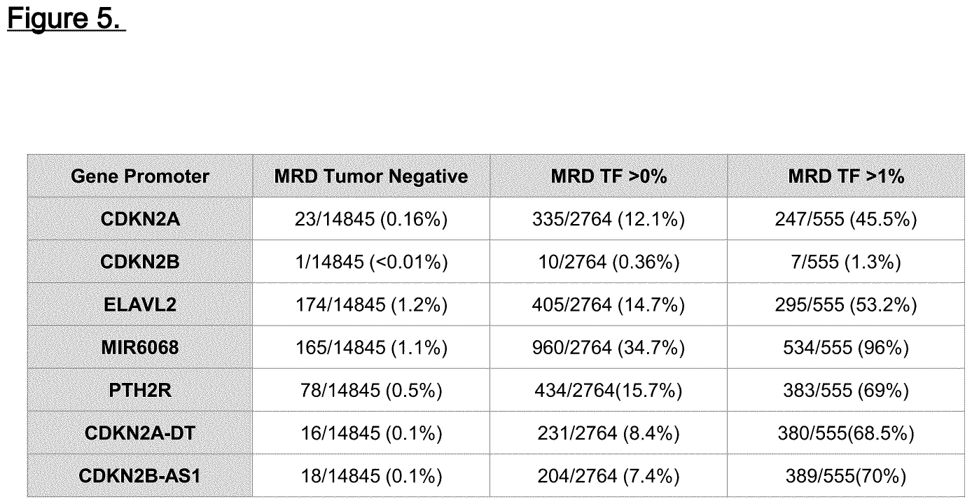

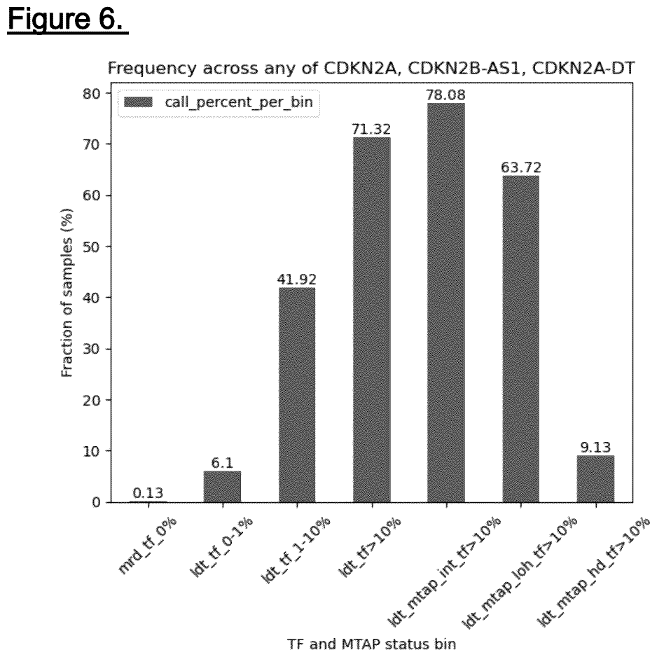

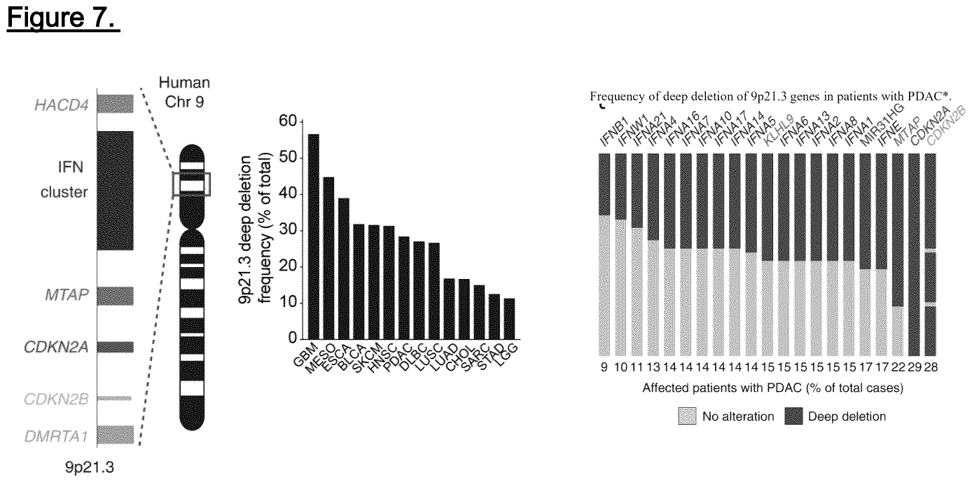

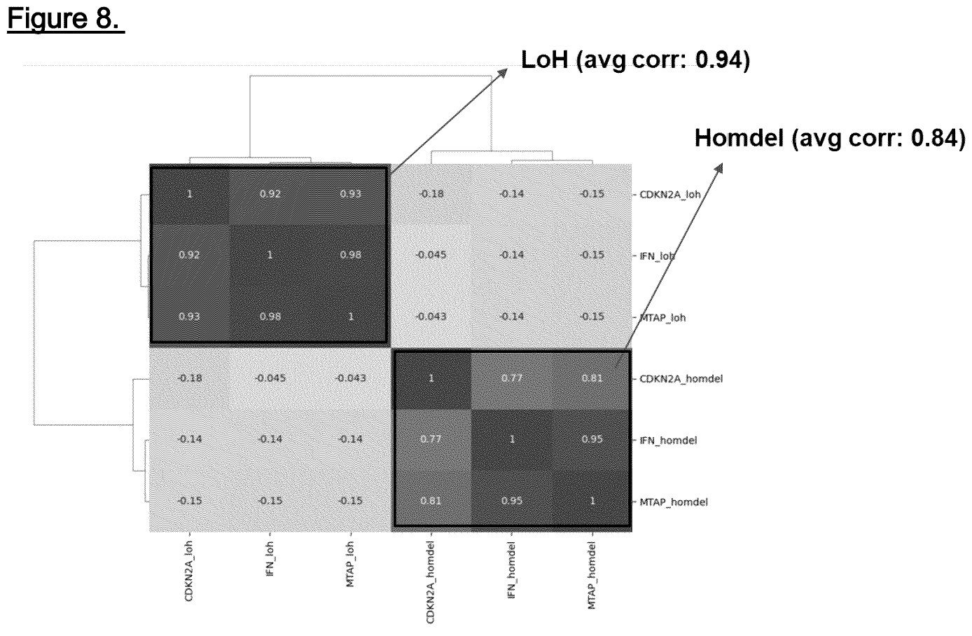

Prediction of MTAP HomDel via tumor-specific methylation from tissue biopsies demonstrates good accuracy and detects MTAP HomDel that conventional coverage-based methods miss in low tumor cellularity/purity samples. As illustrated in FIG. 5, matched tissue samples with sufficient tumor purity can detect HomDel via conventional genomic methods, but matched liquid samples with low tumor fraction (e.g., 2.4%) fail with conventional methods while succeeding with the tumor-specific methylation approach.

Analytical accuracy was assessed using 87 matched cfDNA and tissue samples from patients with advanced cancer, including NSCLC (40%), pancreatic (9%), melanoma (9%), breast (7%), and other cancer types (35%). MTAP HomDel and single-copy number loss were treated as distinct categories.

The multi-omic methodology achieved positive predictive value (PPV) of 100% and negative percent agreement (NPA) of 100% above the limit of detection (90.9% and 96.6% overall) against a tissue-based reference.

As shown in FIG. 9, the validation study demonstrates strong concordance between the methylation-enhanced method and tissue NGS as an orthogonal truth for MTAP HomDel status.

### Example 9—EGFR Driver Mutation Prediction From Tissue

Tissue biopsy samples were analyzed using methylation profiling to predict the presence of EGFR driver mutations. TCGA 450K array data was used to train models for detecting EGFR Exon 19 deletions, L858R, G719, and L861Q variants.

The methylation-based EGFR mutation prediction model demonstrated promising performance, correctly identifying samples with EGFR driver mutations based solely on methylation patterns. This demonstrates the broader applicability of methylation-based prediction beyond gene deletions to include point mutations and small indels.

1. A method, comprising:
generating a methylation profile for at least one nucleic acid sequence obtained from a human subject, wherein the methylation profile comprises at least one differentially menthylated region (DMR).

2. The method of claim 1, wherein the at least one DMR is determined based on a comparison to a threshold determined from one or more healthy subjects, optionally comprising a DMR that is tumor-specific.

3. The method of claim 1, wherein the methylation profile characterizes a gain of function for at least one or more genes.

4. The method of claim 1, wherein the methylation profile characterizes a loss of function for at least one or more genes.

5. The method of claim 1, wherein the methylation profile characterizes a genomic mutation.

6. The method of claim 1, wherein the methylation profile characterizes a genetic target, optionally comprising a genetic variant.

7. The method of claim 1, wherein the methylation profile characterizes gene dysfunction.

8. The method of claim 7, wherein the characterization is based on hypermethylation status.

9. The method of claim 1, comprising obtaining nucleic acid from a biological sample comprising cell-free DNA or tissue DNA.

10. The method of claim 1, comprising determining tumor-specific methylation status at a plurality of loci proximal to a genomic mutation, genetic target, optionally comprising genetic variant.

11. The method of claim 1, comprising determining tumor-specific methylation status at a plurality of loci distal to the genetic target.

12. The method of claim 1, comprising applying a classifier that integrates proximal and distal methylation features to generate a probability score for homozygous deletion of the target gene, optionally comprising a trained classifier.

13. The method of claim 1, comprising, classifying the sample as having or not having a genomic mutation, genetic variant, gene dysfunction, based on the probability score.

14. The method of claim 1, comprising, classifying the sample as having or not having homozygous deletion based on the probability score.

15. The method of claim 1, wherein the genetic target is one or more genes selected selected from the group consisting of MTAP, CDKN2A, PTEN, and RB1.

16. The method of claim 1, wherein the biological sample has a tumor fraction of less than 1%, less than 2%, less than 3%, less than 5%, less than 10%.

17. The method of claim 1, wherein the biological sample has a tumor fraction of less than 2%.

18. The method of claim 1, trained classifier comprises a logistic regression model with elastic net regularization.

19. The method of claim 1, wherein the plurality of loci proximal to the target gene comprises at least differentially methylated regions within 0.1-0.5, 0.5-1, 2, 3, MB of the target gene.

20. The method of claim 1, wherein the genetic target comprises MTAP and the plurality of loci proximal to MTAP comprises methylated regions adjacent to or within MTAP, CDKN2A, CDKN2A-DT, CDKN2B-AS1, DMRTA1, MIR31HG, and HACD4 genes.

21. The method of claim 11, wherein determining tumor-specific methylation status comprises sequencing bisulfite-converted DNA.

22. The method of claim 12, wherein the methylation features comprise binary indicators of methylation presence or absence, a probabilistic function or other quantitative measure.

23. The method of claim 12, wherein the methylation features comprise quantitative measures of methylation levels.

24.-45. (canceled)

46. A system for detecting gene deletions, comprising: a sequencing platform configured to generate methylation and genomic data from a biological sample; a processor configured to receive methylation data for loci proximal and distal to a target gene; receive genomic copy number data for the target gene; (apply trained classifiers to generate methylation-based and genomics-based probability scores; integrate the probability scores through a weighted ensemble; and output a classification of gene deletion status, optionally comprising a display for presenting the classification to a user.
